(19) United States
(12) Patent Application Publication
(10) Pub. No.: US 2005/0142070 A1
(43) Pub. Date: Jun. 30, 2005

(54) Methods and systems for assessing pulmonary disease with drug therapy control

(76) Inventors: Jesse W. Hartley, Lino Lakes, MN (US); Jeffrey E. Stahmann, Ramsey, MN (US); Kent Lee, Fridley, MN (US); Quan Ni, Shoreview, MN (US)

Correspondence Address: CRAWFORD MAUNU PLLC, 1270 NORTHLAND DRIVE, SUITE 390, ST. PAUL, MN, 55120, US

(21) Appl. No.: 10/943,077

(22) Filed: Sep. 15, 2004

Related U.S. Application Data
(60) Provisional application No. 60/503,808, filed on Sep. 18, 2003.

(51) Int. Cl. A61L 9/04; A61K 9/22

(52) U.S. Cl. 424/45; 604/890.1

(57) ABSTRACT

An external respiratory therapy device incorporates sensors that may be used to sense physiological conditions or parameters associated with pulmonary disease. The sensed conditions may be used to detect and/or to assess a presence of various types of pulmonary diseases. The assessment of the pulmonary disease may be utilized to control a drug therapy delivered to the patient to treat the pulmonary disease.

### RELATED PATENT DOCUMENTS

This application claims the benefit of Provisional Patent Application Ser. No. 60/503,808, filed on Sep. 18, 2003, to which priority is claimed pursuant to 35 U.S.C. §119(e) and which is hereby incorporated herein by reference.

### FIELD OF THE INVENTION

The present invention relates generally to methods and systems for assessing a presence of a pulmonary disease and delivering drug therapy to treat the pulmonary disease.

### BACKGROUND OF THE INVENTION

Diseases and disorders of the pulmonary system are among the leading causes of acute and chronic illness in the world. Pulmonary diseases or disorders may be organized into various categories, including, for example, breathing rhythm disorders, obstructive diseases, restrictive diseases, infectious diseases, pulmonary vasculature disorders, pleural cavity disorders, and others. Pulmonary dysfunction may involve symptoms such as apnea, dyspnea, changes in blood or respiratory gases, symptomatic respiratory sounds, e.g., coughing, wheezing, respiratory insufficiency, and/or general degradation of pulmonary function, among other symptoms.

Breathing rhythm disorders involve patterns of interrupted and/or disrupted breathing. Sleep apnea syndrome (SAS) and Cheyne-Stokes respiration (CSR) are examples of breathing rhythm disorders. Breathing rhythm disorders may be caused by an obstructed airway and/or by derangement of the signals from the brain controlling respiration. Disordered breathing rhythm during sleep is particularly prevalent and is associated with excessive daytime sleepiness, systemic hypertension, increased risk of stroke, angina, and myocardial infarction. Breathing rhythm disorders can be particularly serious for patients concurrently suffering from cardiovascular deficiencies.

Obstructive pulmonary diseases can be associated with a decrease in the total volume of exhaled airflow caused by a narrowing or blockage of the airways. Examples of obstructive pulmonary diseases include asthma, emphysema and bronchitis. Chronic obstructive pulmonary disease (COPD) refers to chronic lung diseases that result in blocked airflow in the lungs. Chronic obstructive pulmonary disease may develop over many years, typically from exposure to cigarette smoke, pollution, or other irritants. Over time, the elasticity of the lung tissue is lost, the lung's air sacs may collapse, the lungs may become distended, partially clogged with mucus, and/or lose the ability to expand and contract normally. As the disease progresses, breathing becomes labored, and the patient grows progressively weaker. Many people with COPD concurrently have both emphysema and chronic bronchitis.

Restrictive pulmonary diseases involve a decrease in the total volume of air that the lungs are able to hold. Often the decrease in total lung volume is due to a decrease in the elasticity of the lungs themselves, or may be caused by a limitation in the expansion of the chest wall during inhalation. Restrictive pulmonary disease can be caused by scarring from pneumonia, tuberculosis, or sarcoidosis. A decrease in lung volume may be the result of various neurologic and/or muscular diseases affecting the neural signals and/or muscular strength of the chest wall and lungs. Examples of neurologic and/or muscular diseases that may affect lung volume include poliomyelitis and multiple sclerosis. Lung volume deficiencies may also be related to congenital or acquired deformities of the chest.

Pulmonary dysfunctions can also involve disorders of the pleural cavity and/or pulmonary vasculature. Pulmonary vasculature disorders may include pulmonary hypertension, pulmonary edema, and pulmonary embolism. Disorders of the pleural cavity include conditions such as pleural effusion, pneumothorax, and hemothorax, for example.

Pulmonary disease may be caused by infectious agents such as viral and/or bacterial agents. Examples of infectious pulmonary diseases include pneumonia, tuberculosis, and bronchiectasis. Non-infectious pulmonary diseases include lung cancer and adult respiratory distress syndrome (ARDS), for example.

Early detection and therapy for pulmonary disease improves the likelihood of successful treatment. Methods and systems for detecting and providing therapy for pulmonary diseases and disorders are desirable.

### SUMMARY OF THE INVENTION

Embodiments of the invention involve assessing a presence of a pulmonary disease or disorder that is not a breathing rhythm disorder and controlling the deliver of a drug therapy to treat the pulmonary disease. According to one embodiment, a method for controlling therapy for a non-rhythm related pulmonary disease includes sensing one or more conditions associated with the non-rhythm pulmonary disease using sensors of a patient-external respiratory therapy device. A presence of the non-rhythm pulmonary disease is assessed based on the one or more sensed conditions. A control signal for controlling a drug therapy to treat the non-rhythm pulmonary disease is generated based on the assessment of the non-rhythm pulmonary disease.

According to one aspect, sensing the one or more conditions associated with the non-rhythm pulmonary disease involves performing a pulmonary function test using the sensors of the respiratory therapy device. One or more pulmonary function conditions are determined based on the pulmonary function test.

According to another aspect, the method includes comprising delivering the drug therapy using the generated control signal. In one embodiment, the drug therapy may be delivered using the respiratory therapy device. In other embodiments, the drug therapy may be delivered using a therapy device other than the respiratory therapy device.

One or more additional conditions associated with the non-rhythm pulmonary disease may be sensed using an implantable device. The disease assessment may be based in part on the one or more additional conditions.

Another embodiment of the invention involves a medical system for controlling therapy to treat a non-breathing rhythm related pulmonary disease. The system includes an external respiratory therapy device. The external respiratory therapy device includes a therapy unit configured to deliver respiration therapy to a patient and a sensor system configured to sense one or more conditions associated with a non-rhythm pulmonary disease. A diagnosis unit is coupled to the sensor system and is configured to assess a presence of the non-rhythm pulmonary disease based on the one or more sensed conditions. A drug therapy controller is coupled to the diagnosis unit. The drug therapy controller is configured to control a drug therapy delivered to the patient to treat the non-rhythm pulmonary disease.

The above summary of the present invention is not intended to describe each embodiment or every implementation of the present invention. Advantages and attainments, together with a more complete understanding of the invention, will become apparent and appreciated by referring to the following detailed description and claims taken in conjunction with the accompanying drawings.

### BRIEF DESCRIPTION OF THE DRAWINGS

FIG. 1A is a flowchart illustrating a method of determining a presence of a non-rhythm pulmonary disease and delivering therapy in accordance with embodiments of the invention;

FIGS. 1B-1D are graphs of normal, obstructive and restrictive respiratory patterns, respectively, in accordance with embodiments of the invention;

FIG. 1E is a block diagram of a medical system that includes components useful in implementing detection and/or assessment of non-rhythm pulmonary diseases and controlling drug therapy in accordance with embodiments of the invention;

FIGS. 2A-2D are block diagrams systems that may be used for control of drug therapy in accordance with embodiments of the invention;

FIGS. 3A-3G illustrate a chart depicting relationships between pulmonary diseases, symptoms and/or physiological changes caused by the pulmonary diseases, and conditions used to detect the symptoms and/or physiological changes in accordance with embodiments of the invention;

FIG. 4A is a partial view of an implantable medical device that may be used for medical disease/disorder detection and/or drug therapy control in accordance with embodiments of the invention;

FIG. 4B is an illustration of a thorax having an implanted subcutaneous medical device that may be used for medical disease/disorder detection and/or drug therapy control in accordance with embodiments of the present invention; and

FIG. 5 is a flowchart illustrating a method of assessing a presence of a non-rhythm pulmonary disease and delivering drug therapy in accordance with embodiments of the invention.

While the invention is amenable to various modifications and alternative forms, specifics thereof have been shown by way of example in the drawings and will be described in detail below. It is to be understood, however, that the intention is not to limit the invention to the particular embodiments described. On the contrary, the invention is intended to cover all modifications, equivalents, and alternatives falling within the scope of the invention as defined by the appended claims.

### DETAILED DESCRIPTION OF VARIOUS EMBODIMENTS

In the following description of the illustrated embodiments, references are made to the accompanying drawings which form a part hereof, and in which are shown by way of illustration, various embodiments by which the invention may be practiced. It is to be understood that other embodiments may be utilized, and structural and functional changes may be made without departing from the scope of the present invention.

Pulmonary disorders may be organized into broad categories encompassing disorders of breathing rhythm and non-rhythm pulmonary diseases and/or disorders. Breathing rhythm disorders include various syndromes characterized by patterns of disordered breathing that produce insufficient respiration, for example, sleep apnea, hypopnea, and Cheyne-Stokes Respiration (CSR), among others. Breathing rhythm disorders are not necessarily accompanied by alteration of pulmonary structures.

Non-rhythm pulmonary diseases or disorders typically involve physical changes to lung structures, such as loss of elasticity of the lung tissue, obstruction of airways with mucus, limitation of the expansion of the chest wall during inhalation, fibrous tissue within the lung, excessive pressure in the pulmonary arteries, and/or other characteristics. Pulmonary diseases or disorders that are not rhythm-related are referred to herein as non-rhythm pulmonary diseases and may include various types, for example, obstructive pulmonary diseases, restrictive pulmonary diseases, infectious and non-infectious pulmonary diseases, pulmonary vasculature disorders, and pleural cavity disorders.

Embodiments of the invention are directed to controlling a drug therapy to treat a non-rhythm pulmonary disease. A presence of a non-rhythm pulmonary disease is determined using a sensor system coupled to a respiratory therapy device. If the non-pulmonary disease is present based on the assessment, then a drug therapy to treat the non-pulmonary disease may be delivered. In accordance with embodiments of the invention, a non-rhythm pulmonary disease assessment system may be used to discriminate between types of non-rhythm pulmonary diseases, e.g., between obstructive pulmonary diseases and restrictive pulmonary diseases. The non-rhythm pulmonary disease assessment system may discriminate between non-rhythm pulmonary diseases of a particular type, e.g., between asthma and emphysema, both of which are pulmonary diseases of the obstructive type. Discrimination between pulmonary diseases afflicting the patient facilitates delivery of an effective drug therapy, allowing the system to deliver an appropriate therapy for the particular pulmonary disease detected.

If the presence of a non-rhythm pulmonary disease is determined, then the progression of the disease may be monitored. Monitoring the progression of the non-rhythm pulmonary disease may involve, for example, periodically evaluating one or more physiological changes or symptoms associated with the disease. Evaluation of the one or more physiological changes or symptoms may be accomplished by sensing conditions associated with the symptoms or physiological changes. In a preferred embodiment, information about the sensed conditions is stored and may be trended or otherwise processed to facilitate disease detection.

As referenced herein, the term “condition,” denotes an attribute that may be sensed and/or measured based on a signal generated by a sensor or other input device of a respiratory therapy device or another medical device. Typically, a physiological sensor generates a signal modulated by a physiological parameter. In some cases, a physiological condition may be directly measured based on the sensor signal. For example, a blood pressure measurement may directly correlate to the signal generated by a calibrated blood pressure sensor. In other cases, a condition measurement may be derived from the sensor signal. For example, tidal volume is a respiratory system condition that may be derived based on the signal generated by a transthoracic impedance sensor. In another example, heart rate is a cardiac system condition that may be derived from a cardiac electrogram sensor.

The terms “symptom” and “physiological change” refer to a manifestation of a medical disease or disorder. Symptoms and/or physiological changes may be detectable based on a sensed presence of one or more physiological conditions and/or measured values associated with the one or more sensed physiological conditions. The terms “disease” and/or “disorder” are used to refer to a medical dysfunction that is characterizable by a collection of symptoms or physiological changes.

Monitoring a disease may involve, for example, monitoring the severity and/or other characteristics of the disease over time. Monitoring the disease may involve detecting disease onset, monitoring progression and/or regression of the disease and detecting disease offset. Disease monitoring may involve monitoring one or more conditions associated with the physiological changes and/or symptoms of the disease.

In one implementation, the presence of the non-rhythm pulmonary disease is assessed based on one or more patient conditions indicative of symptoms or physiological changes associated with the disease. The one or more conditions are sensed using the sensing system of a patient-external respiratory therapy device. The respiratory therapy device may comprise, for example, a gas therapy device, nebulizer, ventilator, positive airway pressure device, or other type of respiration therapy device. In a preferred embodiment, the respiratory therapy device comprises a positive airway pressure device.

Continuous positive airway pressure (CPAP) devices are frequently used to treat sleep apnea and/or other breathing rhythm disorders. A CPAP device may be used regularly during a patient's sleep time to alleviate symptoms of breathing rhythm related disorders. The sensors of the CPAP device, used nightly to treat disordered breathing disorders, may be employed to detect and/or assess non-rhythm pulmonary diseases. A drug therapy for the non-rhythm pulmonary disease may be controlled based on the assessment of the disease.

In another implementation, the presence of the non-rhythm pulmonary disease may be detected and/or assessed based on conditions sensed using sensors of a patient-external respiratory therapy device in combination with additional conditions sensed using sensors of an implantable device. The implantable device may comprise, for example, an implantable cardiac device, such as a pacemaker, defibrillator, cardioverter, cardiac monitor, and/or cardiac resynchronizer.

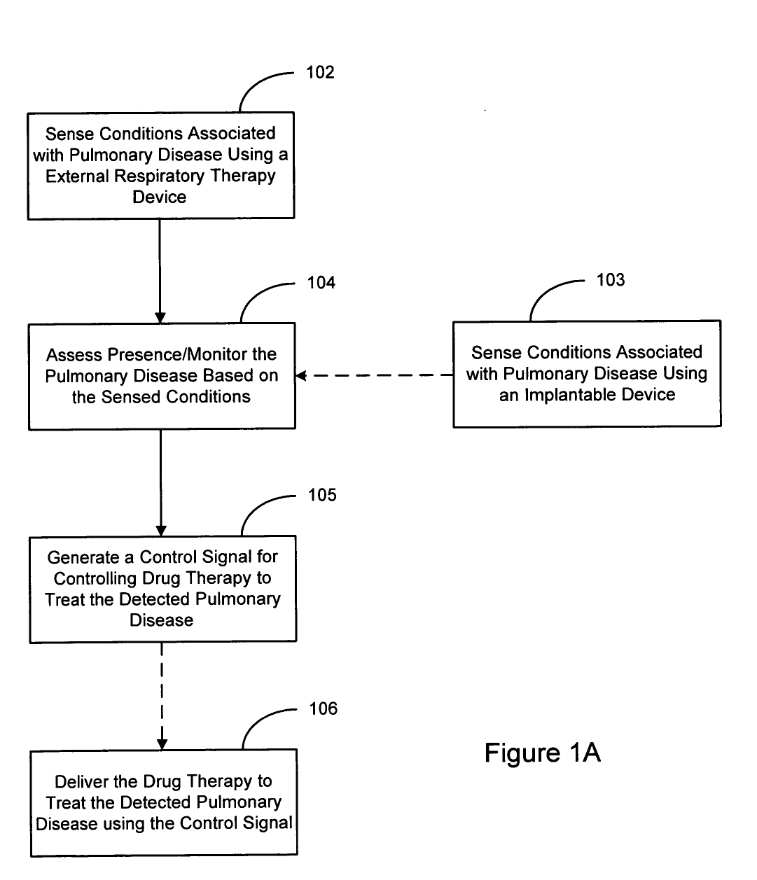

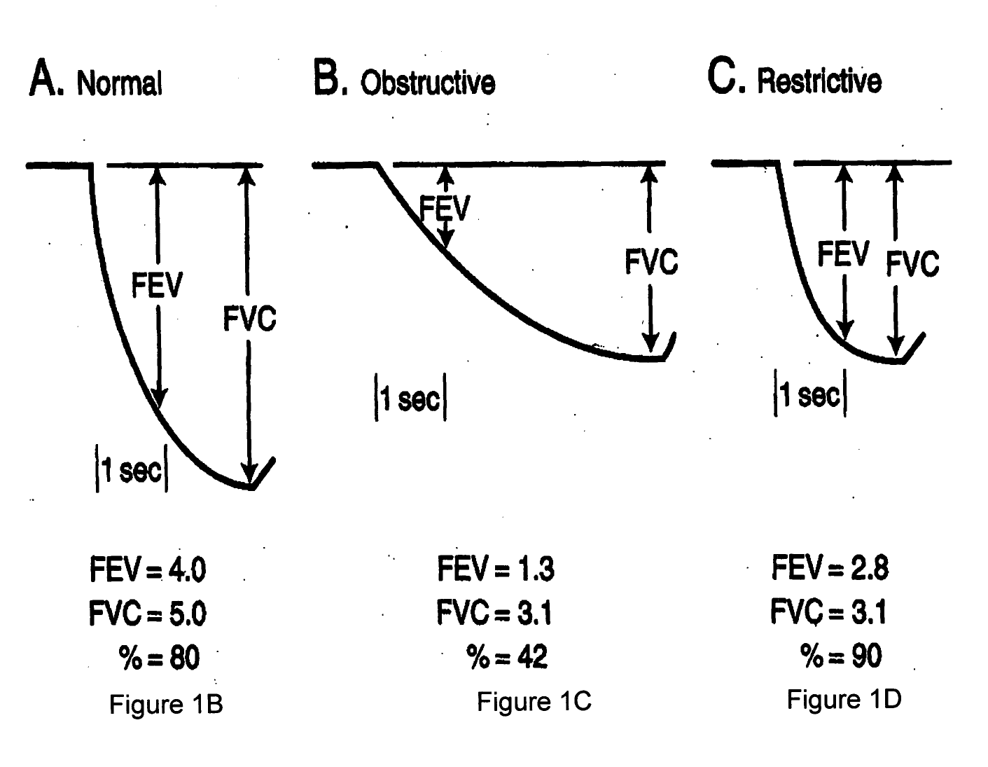

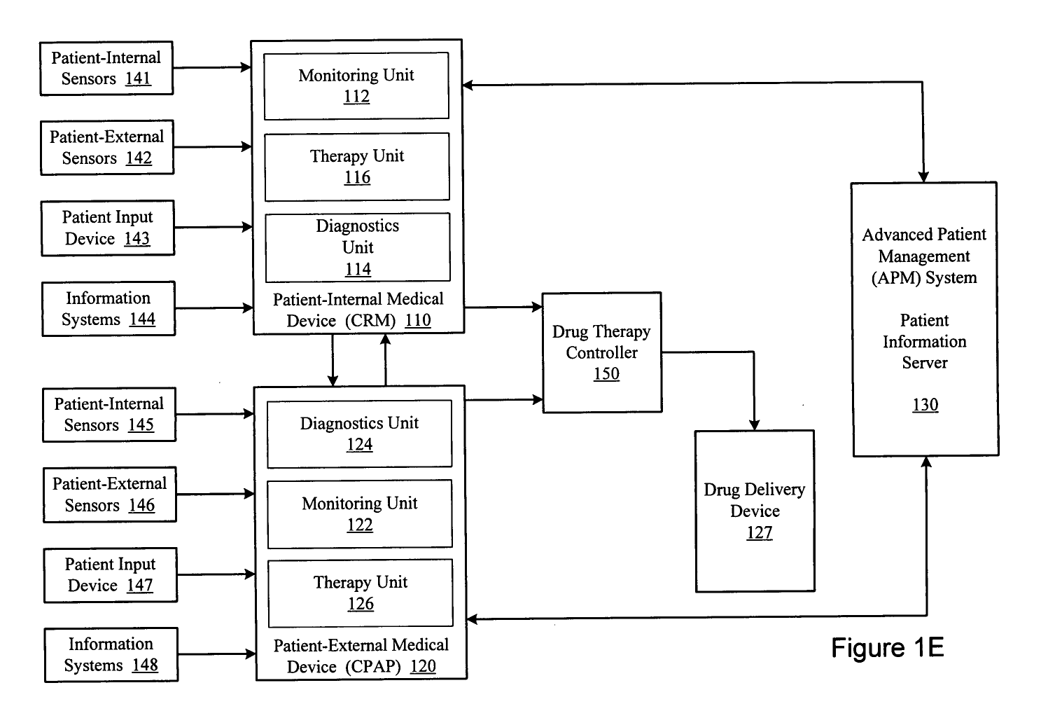

FIG. 1A is a flowchart illustrating various optional methods for controlling drug therapy in accordance with embodiments of the invention. In some embodiments, the system generates a control system for controlling the drug therapy. In other embodiments, the system includes a drug delivery unit that is controlled by the control signal.

One method involves using 102 an external respiratory therapy device to sense conditions associated with the non-rhythm related pulmonary disease. A presence of the non-rhythm pulmonary disease is assessed 104 based on the sensed conditions. A control signal for controlling drug therapy used to treat the detected pulmonary disease is generated 105. Approaches to assessing the presence of a non-rhythm pulmonary disease, aspects of which may be utilized in connection with embodiments of the present invention, are provided in commonly owned U.S. Patent Application identified by Attorney Docket No.: GUID.136PA, entitled “Methods and Systems for Assessing Pulmonary Disease,” filed Aug. 31, 2004, and incorporated herein by reference.

Optionally, the external respiratory therapy device may sense 102 one set of conditions and an implantable device may be used to sense 103 another set of conditions. The disease presence may be assessed based on the conditions sensed by the external respiratory therapy device and the conditions sensed by the implantable device. In one implementation, the external respiratory therapy device and the implantable device may be used cooperatively to sense conditions affecting the patient and to detect and/or assess a disease presence. Cooperative use of medical devices for assessing medical disorders, aspects of which may be utilized in connection with various embodiments described herein, are discussed in commonly owned U.S. Patent Application, identified by Attorney Docket No.: GUID.126PA, entitled “Synergistic Use of Medical Devices for Detecting Medical Disorders,” filed concurrently with this patent application, and incorporated herein by reference.

In some embodiments the system includes a drug delivery device. The drug delivery device delivers 106 a drug therapy that is controlled by the control signal. The drug delivery device may be a component of the external respiratory therapy device, the implanted device, or a device separate from the external respiratory therapy device and the implanted device. The drug delivery device may comprise a drug pump, an activatable drug patch, and/or a gas therapy delivery device, for example.

In one scenario, the respiratory device is a CPAP device that has drug delivery functionality. Upon detection and/or assessment of a non-rhythm pulmonary disease, such as asthma, the CPAP device can activate a drug delivery unit to deliver a mist, e.g., an albuterol mist, into the air stream supplied by the CPAP device.

In another scenario, drug therapy may be accomplished using an implantable drug delivery device such as an implantable drug pump. In one implementation, the implantable drug delivery device is configured as a component of an implantable cardiac rhythm management (CRM) system. In another implementation, the implantable drug delivery device is separate from the CRM or other implantable device used for sensing.

Assessing the presence of pulmonary disease may be enhanced by the performance of pulmonary function tests. Pulmonary function testing evaluates lung mechanics, gas exchange, pulmonary blood flow, and blood gases and pH. These tests may be used to evaluate patients in the diagnosis of pulmonary disease and assessment of disease development. According to various aspects of the invention, pulmonary function testing may be implemented using the sensors of the respiratory therapy device, and/or using the sensors of the implantable device.

Pulmonary function testing is conventionally performed in a clinical setting and measures values indicative of the ability of the lungs to exchange oxygen and carbon dioxide. The total lung capacity (TLC) is divided into four volumes. The tidal volume (VT) is the volume inhaled or exhaled in normal quiet breathing. The inspiratory reserve volume (IRV) is the maximum volume that can be inhaled following a normal quiet inhalation. The expiratory reserve volume (ERV) is the maximum volume that can be exhaled following a normal quiet exhalation. The residual volume (RV) is the volume remaining in the lungs following a maximal exhalation. The vital capacity (VC) is the maximum volume that can be exhaled following a maximal inhalation; VC=IRV+V_{T}+ERV. The inspiratory capacity (IC) is the maximum volume that can be inhaled following a normal quiet exhalation; IC=IRV+V_{T}. The functional residual capacity (FRC) is the volume remaining in the lungs following a normal quiet exhalation; FRC=ERV+RV.

The vital capacity and its components (V_{T}, IRV, ERV, IC) are typically measured using a spirometer, which is a device that measures the volumes of air inhaled and exhaled. The FRC is usually measured by the helium dilution method using a closed spirometry system. A known amount of helium is introduced into the system at the end of a normal quiet exhalation. When the helium equilibrates throughout the volume of the system, which is equal to the FRC plus the volume of the spirometer and tubing, the FRC is determined from the helium concentration. This test may underestimate the FRC of patients with emphysema. The FRC can be determined quickly and more accurately by body plethysmography. The residual volume and total lung capacity are determined from the FRC.

In the forced vital capacity (FVC) maneuver, the patient exhales as forcefully and rapidly as possible, beginning at maximal exhalation. Several parameters are determined from the spirogram. The FVC is the total volume of air exhaled during the maneuver; it is normally equal to the vital capacity. The forced expiratory volume (FEV) is the volume expired during a specified time period from the beginning of the test. The times used are 0.5, 1, 2, and 3 seconds; corresponding parameters are FEV_{0.5}, FEV_{1.0}, FEV_{2.0}, and FEV_{3.0}. The maximal expiratory flow rate (MEFR) is the slope of the line connecting the points where 200 ml and 1200 ml have been exhaled; it is also called FEF₂₀₀₋₁₂₀₀ (forced expiratory flow). The maximal midexpiratory flow rate (MMFR, MMF) is the slope of the line connecting the points where 25 percent and 75 percent of the FVC have been exhaled; it is also called FEF_{25-75%}.

The Maximal Voluntary Ventilation (MW) is the maximal volume of air that can be breathed by the patient, expressed in liters per minute; it was formerly called maximal breathing capacity (MBC). The patient breathes as rapidly and deeply as possible for 12 to 15 seconds and the volume exhaled is determined by spirometry.

Various parameters related to pulmonary performance, some of which may be measured using sensors of a respiratory therapy device and/or sensors of an implantable device include, for example, tidal volume, minute ventilation, inspiratory reserve volume, forced expiratory volume, residual volume, and forced vital capacity, among other parameters. According to one embodiment, testing of some pulmonary function parameters may be performed using the ventilation pressure and ventilation flow sensors of a CPAP device or other patient-external respiratory therapy device. The pulmonary function testing may be used, for example, to assess a presence of restrictive and/or obstructive pulmonary disorders as indicated in FIGS. 1B-1D.

Pulmonary performance may be evaluated based on data acquired by the respiratory therapy device during normal and forced inspiration and expiration. From such data, pulmonary parameters including tidal volume, minute ventilation, forced expiratory volume, forced vital capacity, among other parameters may be determined.

Because the results of pulmonary function tests vary with size and age, the normal values are calculated using prediction equations or nomograms, which give the normal value for a specific age, height, and sex. The prediction equations are derived using linear regression on the data from a population of normal subjects. The observed values are usually reported as a percentage of the predicted value. Abnormal test results may show either an obstructive or restrictive pattern. Sometimes, both patterns are present.

FIG. 1B illustrates a normal respiratory pattern, having normal FEV and FVC. FIG. 1C illustrates an obstructive pattern. An obstructive pattern occurs when there is airway obstruction from any cause, as in asthma, bronchitis, emphysema, or advanced bronchiectasis; these conditions are grouped together in the nonspecific term chronic obstructive pulmonary disease (COPD). In this pattern, the residual volume is increased and the RV/TLC ratio is markedly increased. Owing to increased airway resistance, the flow rates are decreased. The FEV/FVC ratios, MMFR, and MEFR are all decreased; FEV_{1.0}/FVC is less than 75 percent.

FIG. 1D illustrates a restrictive pattern. A restrictive pattern occurs when there is a loss of lung tissue or when lung expansion is limited as a result of decreased compliance of the lung or thorax or of muscular weakness. The conditions in which this pattern can occur include pectus excavatum, myasthenia gravis, diffuse idiopathic interstitial fibrosis, and space occupying lesions (tumors, effusions). In this pattern, the vital capacity and FVC are less than 80 percent of the predicted value, but the FEV/FVC ratios are normal. The TLC is decreased and the RV/TLC ratio is normal.

Embodiments of the invention utilize a patient-external respiratory therapy device to perform periodic pulmonary function testing. A CPAP or other external respiratory device may measure ventalitory pressure, ventilatory airflow, and/or ventalitory gas concentration during periodic, e.g., nightly, therapy sessions. The ventalitory pressure and/or airflow measurements may be used to measure FVC and FEV during forced expiration. From these two parameters, FEV/FVC can be derived to differentiate obstructive versus restrictive respiratory patterns as shown in the FIGS. 1C and 1D. Other measurements that are possible using the respiratory device sensors include low forced expiratory flow (FEF), high functional residual capacity (FRC), total lung capacity (TLC), and high residual volume (RV).

In one embodiment, the patient may perform forced expirations while connected to the external respiratory device. During the forced expirations, circuitry in the external respiratory device may collect measurements, including measurements useful in calculating the FEV and FVC measurements.

In addition, the forced expiratory flow (FEF_{25-75%}) may be measured. The middle half by volume of the total expiration is marked, and its duration is measured. The FEF_{25-75%} is the volume in liters divided by the time in seconds. In patients with obstructive diseases, the FEF_{25-75%} is generally greater than their expected values.

Circuitry incorporated in the CPAP device may be used to compare measured FVC, FEV and FEF_{25-75%} values derived from the respiratory therapy device pressure sensors and/or airflow sensors with predicted values from normal subjects in accordance with various embodiments. The comparison provides diagnostic information of lung mechanics. Data acquired by the CPAP device may be transmitted, for example, from the respiratory therapy device to an advanced patient management (APM) system or other remote device.

In some embodiments, pulmonary function testing may be performed using a cardiac rhythm management system (CRM) or other implantable device. In one implementation, the pulmonary function testing is performed using an implanted transthoracic impedance sensor. Transthoracic impedance sensing has been used in connection with rate-adaptive pacemakers to measure respiration cycles. An impedance sensor may be used to measure the variation in transthoracic impedance, which increases during the inspiratory and decreases during the expiratory phase of a respiration cycle. The sensor injects a sub-threshold stimulating current between the pacemaker case and an electrode on an intracardiac or subcutaneous lead, and measures the voltage across the case and another electrode on the same or another lead. Clinical investigations have shown that the impedance sensor can measure respiratory rate tidal volume, and minute ventilation accurately.

In accordance with various embodiments of the invention, a properly calibrated impedance sensor, implemented in cooperation with a pacemaker or other implantable device, may be used to measure FVC and FEV during forced expiration. From these two parameters, FEV/FVC can be derived to differentiate obstructive versus restrictive respiratory patterns as shown in the FIGS. 1C and 1D, respectively.

In addition, the forced expiratory flow (FEF_{25-75%}) may be measured. The middle half by volume of the total expiration is marked, and its duration is measured. The FEF_{25-75%} is the volume in liters divided by the time in seconds. In patients with obstructive diseases, the FEF_{25-75%} is generally greater than their expected values.

The implantable device may be used to compare measured FVC, FEV and FEF_{25-75%} values derived from the implanted impedance sensor with predicted values from normal subjects in accordance with various embodiments. The comparison provides diagnostic information of lung mechanics. Data acquired using the above-described techniques may be transmitted from the implantable device to an advanced patient management system or other remote device. Assessment of the patient's cardiopulmonary status or control of the therapy may be performed by the advanced patient management system.

Methods and systems for acquiring and using pulmonary function testing information, aspects of which may be utilized in connection with embodiments of the invention, are described in commonly owned U.S. patent application Ser. No. 10/885,145, filed Jul. 6, 2004, which is incorporated herein by reference.

FIG. 1E is a block diagram of a medical system 100 that includes components useful in implementing detection and/or assessment of non-rhythm pulmonary diseases and controlling drug therapy in accordance with embodiments of the invention. One or more of the components identified in FIG. 1E may be used for assessing pulmonary diseases and controlling delivery of drug therapy. For example, the medical system 100 may be implemented to include one or more of the features and/or processes described herein. A system for assessing pulmonary diseases and controlling delivery of drug therapy need not include all of the features and functions described, but may be implemented to include one or more selected features and functions that provide unique structures and/or functionality.

FIG. 1E illustrates a patient internal device 110 and a patient external device 120. Sensors, input devices, and/or information systems 141-148 coupled to the patient-internal device and the patient-external device may be used to acquire data related to patient conditions indicative of symptoms of a pulmonary disease. In addition to the sensing functions performed by the therapy devices 110, 120, the devices 110, 120 may respectively include therapy units 116, 126 providing therapy for disorders other than the detected pulmonary disease, e.g., cardiac therapy for cardiac rhythm disorders and/or CPAP therapy for breathing rhythm disorders. Either of the patient internal device 110 and/or the patient external device 120 may include a drug therapy control unit 150 configured to generate control signals deliverable to a drug therapy device 127. The drug therapy device provides drug therapy to treat one or more non-rhythm pulmonary diseases. In some embodiments, the components used to generate the drug therapy control signal or signals may be included in both the patient internal device and the patient external device. The patient internal device 110 and/or the patient external device 120 may generate control signals that initiate, modify, and/or terminate drug therapy for the non-rhythm pulmonary disease.

The patient-internal device 110 is typically a fully or partially implantable device that includes circuitry for implantably performing one or more of monitoring 112, diagnosis 114, and/or therapy control/delivery functions 116, 150. Implantably performing an operation comprises performing the operation using a device that is partially or fully implantable within the patient's body.

The patient-external device 120 includes circuitry for performing one or more of monitoring, diagnosis and/or therapy control/delivery functions patient-externally (i.e., not invasively implanted within the patient's body). The patient-external medical device 120 may be positioned on the patient, near the patient, or in any location external to the patient. It is understood that a portion of a patient-external medical device 120 may be positioned within an orifice of the body, such as the nasal cavity or mouth, yet can be considered external to the patient (e.g., mouth pieces/appliances, tubes/appliances for nostrils, or temperature sensors positioned in the ear canal).

Each of the patient-internal 110 and patient-external 120 devices may include a patient monitoring unit 112, 122. The patient-internal and patient-external devices 110, 120 may be coupled to one or more sensors 141, 142, 145, 146, patient input devices 143, 147 and/or other information acquisition devices 144, 148. The sensors 141, 142, 145, 146, patient input devices 144, 147, and/or other information acquisition devices 144, 148 may be employed to detect conditions relevant to the monitoring, diagnostic, and/or therapeutic functions of the patient-internal and patient-external medical devices 110, 120. The sensors 141, 142, 145, 146, patient input devices 144, 147, and/or other information acquisition devices 144, 148 may be used to detect conditions associated with pulmonary disease.

The medical devices 110, 120 may each be coupled to one or more patient-internal sensors 141, 145 that are fully or partially implantable within the patient. The medical devices 110, 120 may also be coupled to patient-external sensors 142, 146 positioned on, near, or in a remote location with respect to the patient. The patient-internal and patient-external sensors are used to sense conditions, such as physiological or environmental conditions.

The patient-internal sensors 141 may be coupled to the patient-internal medical device 110 through internal leads. In one example, an internal endocardial lead system is used to couple cardiac electrodes that sense cardiac electrical activity to an implantable pacemaker or other cardiac rhythm management device. In some applications, one or more patient-internal sensors 141 may be equipped with transceiver circuitry to support wireless communications between the one or more patient-internal sensors 141 and the patient-internal medical device 110. Similarly, patient internal sensors 145 may be coupled to a patient-external device 120 through wireless communications links.

The patient-external sensors 142, 146 may be coupled to the patient-internal medical device 110 and/or the patient-external medical device 120 through leads or through wireless connections. Patient-external sensors 142 preferably communicate with the patient-internal medical device 110 wirelessly. Patient-external sensors 146 may be coupled to the patient-external medical device 120 through leads or through a wireless link.

The medical devices 110, 120 may be coupled to one or more patient-input devices 143, 147. The patient-input devices are used to allow the patient to manually transfer information to the medical devices 110, 120. The patient input devices 143, 147 may be particularly useful for inputting information concerning patient perceptions, such as how well the patient feels, and information such as patient smoking, drug use, or other activities that are not automatically sensed or detected by the medical devices 110, 120.

The medical devices 110, 120 may be connected to one or more information systems 144, 148, for example, a database that stores information useful in connection with the monitoring, diagnostic, or therapy functions of the medical devices 110, 120. For example, one or more of the medical devices 110, 120 may be coupled through a network to a information system server that provides information about environmental conditions affecting the patient, e.g., the pollution index for the patient's location.

The medical devices 110, 120 may incorporate therapy units 116, 126 for configured to control and deliver therapy to the patient. The therapy units 116, 126 may be implemented to provide therapy other than a drug therapy delivered to treat the pulmonary disease. For example, in one embodiment, the patient-internal device 110 may comprise a cardiac rhythm management (CRM) system configured to deliver cardiac pacing therapy to the patient. The patient-external device 120 may comprise a positive airway pressure (xPAP) device configured to deliver a respiratory therapy to treat a breathing rhythm disorder. One or both of the patient-internal device 110 and the patient-external device 120 may include components that control delivery of a drug therapy to treat the non-rhythm pulmonary disease.

The system 100 further includes a diagnostics unit 114, 124 that is configured to detect and/or assess a presence of non-rhythm pulmonary disease. In some embodiments, the diagnostics unit 124 may be fully incorporated into the patient-external device 120. In other embodiments, the diagnostics unit 114 may be fully incorporated into the patient-internal device 110. In yet other embodiments, components of the diagnostics unit 114 may be incorporated into both the patient-internal and patient-external devices 110, 120. In yet further embodiments, the diagnostics unit may be located remotely from both the patient-internal medical device 110 and the patient-external medical device 120. In one scenario, the diagnostics processor may be implemented as a component of an advanced patient management (APM) system 130, for example.

The monitoring units 112, 122 of the patient-internal and patient external medical devices 110, 120 collect data based on conditions sensed or detected through the use of the sensors 141, 142, 145, 146, patient input devices 143, 146, and/or information systems 144, 148 coupled to the patient-internal and patient-external devices 110, 120. The collected data is transferred to a diagnostics unit 114.

The diagnostics unit 114 is configured to assess the presence of the non-rhythm pulmonary disease based on the sensed conditions. The diagnostics processor 114 may also assess and/or monitor the progression, of the medical disease or disorder. Monitoring the progression of the disease may involve, for example, periodically evaluating one or more conditions indicative of physiological changes or symptoms of the disease. Monitoring disease progression may involve, for example, monitoring the severity of the disease, monitoring disease onset, progression, regression and offset, and/or monitoring other aspects of the disease.

A drug therapy controller 150 may be configured as a component of the patient-internal device 110, the patient external device 120, a device remote from the patient-internal and patient external devices 110, 120, or as a stand alone unit. In some configurations, components of the drug therapy controller may be housed in both the patient-internal and patient external devices 110, 120. Components of the drug therapy controller and the drug therapy delivery unit may be disposed within a single housing.

The drug therapy controller 150 generates a control signal for controlling drug therapy delivered to the patient based on the assessment of the non-rhythm pulmonary disease. The drug therapy controller 150 may generate a control signal to initiate drug therapy if disease onset is detected or if one or more symptoms of the disease are determined to reach a threshold limit, for example. The control signal may indicate termination of the drug therapy if one or more symptoms of the disease subside. Further, during the course of the disease, the control signal may be adjusted based on the assessment of the presence of the non-rhythm pulmonary disease as indicated by sensed conditions indicative of disease symptoms.

The control signal generated by the drug therapy controller 150 is received by the drug therapy unit 127. The drug therapy unit 127, which may comprise an implantable or patient-external device, provides a drug therapy to treat the non-rhythm pulmonary disease. Therapy delivered by the drug therapy unit 127 is controlled by the control signal generated by the drug therapy controller 150. In various embodiments, the drug therapy unit may be implemented as an implantable or patient-external drug pump, a gas therapy device, nebulizer, and/or an activatable drug patch.

In various embodiments, the patient-internal device 110, the patient-external device 120, drug controller 150, drug delivery unit 127, and/or other devices depicted in FIG. 1E may communicate through wireless links. For example, two or more devices, such as the patient-internal and patient-external devices 110, 120, may be coupled through a short-range radio link, such as Bluetooth or a proprietary wireless link. The wireless communications link may facilitate unidirectional or bidirectional communication between the patient-internal 110 and patient-external 120 medical devices. In one implementation, data and/or control signals may be transmitted between the patient-internal 110 and patient-external 120 medical devices to coordinate the functions of the medical devices 110, 120.

In an embodiment of the invention, the patient-internal and patient-external medical devices 110, 120 may be used within the structure of an advanced patient management system. Advanced patient management systems involve a system of medical devices that are accessible through various communications technologies. For example, patient data may be downloaded from one or more of the medical devices periodically or on command, and stored at a patient information server. The physician and/or the patient may communicate with the medical devices and the patient information server, for example, to acquire patient data or to initiate, terminate or modify therapy.

In the implementation illustrated in FIG. 1E, the patient-internal device 110 and the patient-external device 120 may be coupled through a wireless or wired communications link to a patient information server that is part of an advanced patient management (APM) system 130. The APM patient information server 130 may be used to download and store data collected by the patient-internal and patient-external devices 110, 120.

The data stored on the APM patient information server 130 may be accessible by the patient and the patient's physician through terminals, e.g., remote computers located in the patient's home or the physician's office. The APM patient information server 130 may be used to communicate to one or more of the patient-internal and patient-external medical devices 110, 120 to effect remote control of the monitoring, diagnosis, and/or therapy functions of the medical devices 110, 120.

In one scenario, the patient's physician may access patient data transmitted from the medical devices 110, 120 to the APM patient information server 130. After evaluation of the patient data, the patient's physician may communicate through one or more of the patient-internal or patient-external devices 110, 120 through the APM system 130 to initiate, terminate, or modify the monitoring, diagnostic, and/or therapy functions of the patient-internal and/or patient-external medical systems 110, 120. Systems and methods involving advanced patient management techniques, aspects of which may be utilized in connection with a medical disorder detection system in accordance with embodiments of the invention, are further described in U.S. Pat. Nos. 6,336,903, 6,312,378, 6,270,457, 6,398,728, and 6,440,066 which are incorporated herein by reference.

The patient-internal and patient-external medical devices 110, 120 may not communicate directly, but may communicate indirectly through the APM system 130. In this embodiment, the APM system 130 may operate as an intermediary between two or more of the medical devices 110, 120. For example, data and/or control information may be transferred from one of the medical devices 110, 120 to the APM system 130. The APM system 130 may transfer the data and/or control information to another of the medical devices 110, 120.

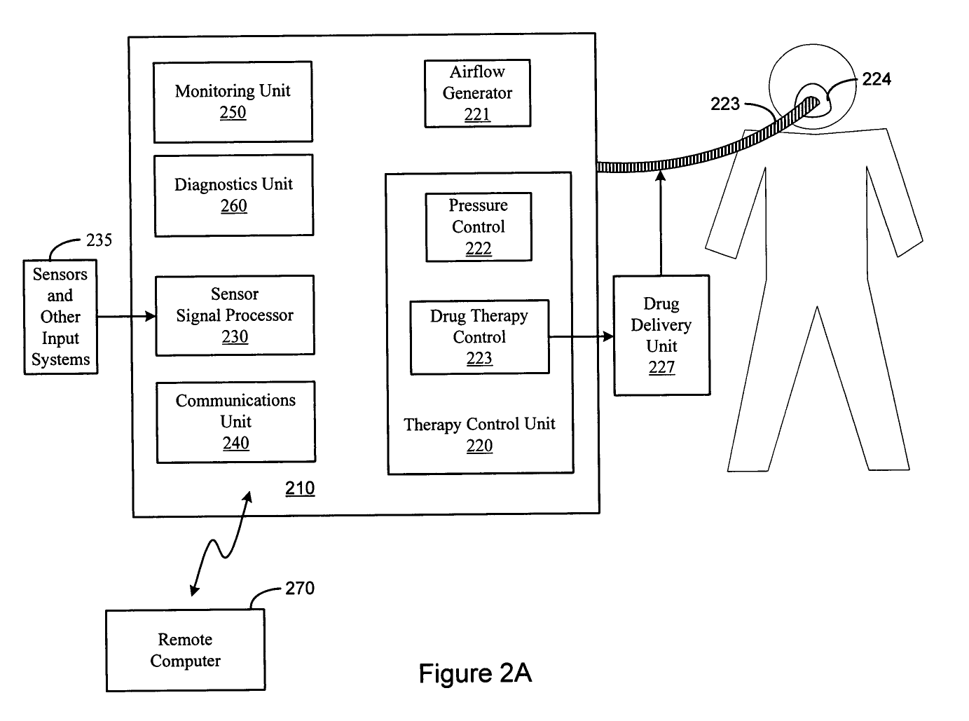

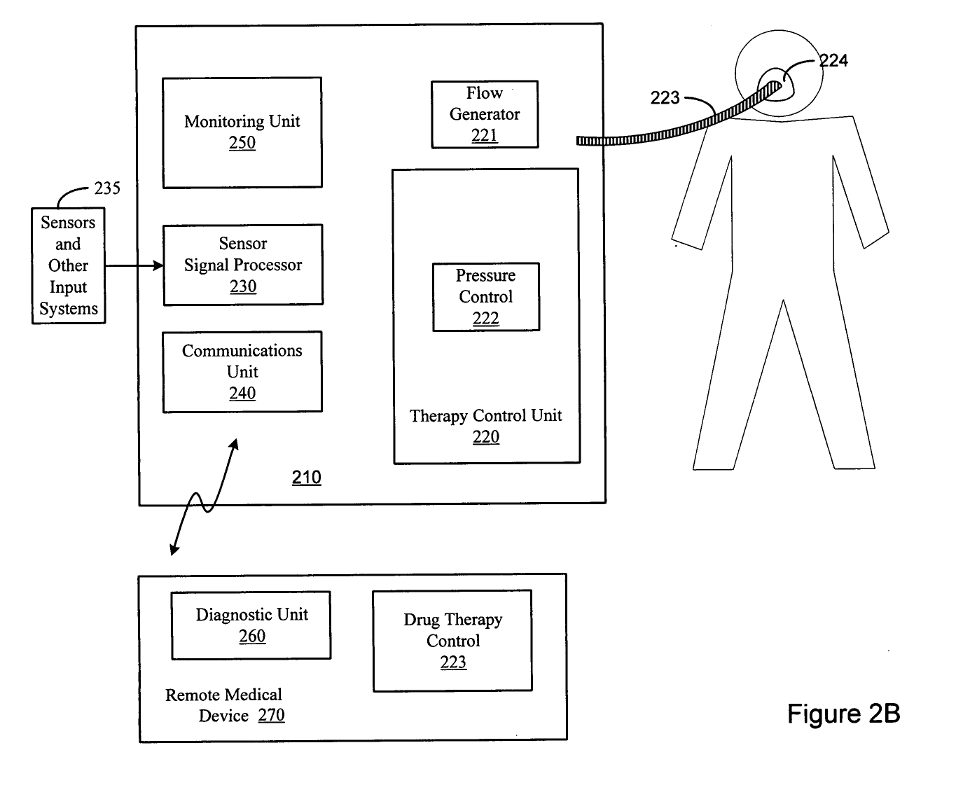

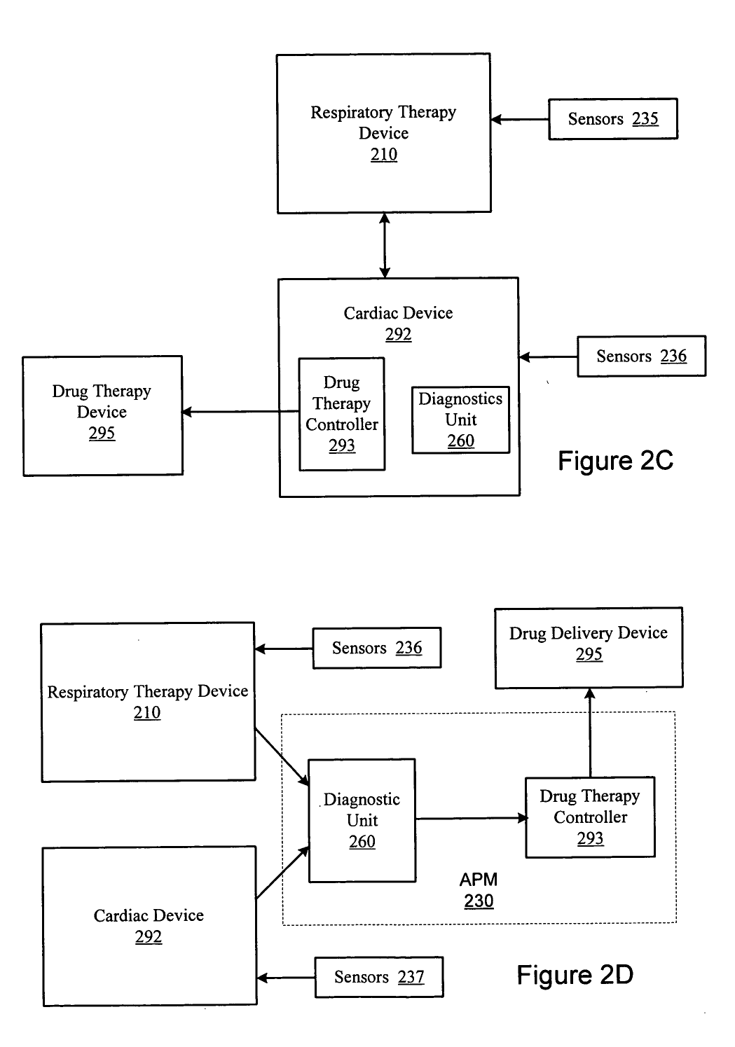

FIGS. 2A-2D are block diagrams of systems that may be used for non-rhythm pulmonary disease assessment with drug therapy control in accordance with embodiments of the invention. FIG. 2A illustrates an external respiratory therapy device 210, e.g., a CPAP device, used to sense conditions associated with a non-rhythm pulmonary disease. The sensed conditions are evaluated by the external respiratory therapy device to assess a presence of the non-rhythm pulmonary disease.

The respiratory therapy device 210 is coupled to one or more sensors or other input devices 235 configured to sense or detect conditions indicative of physiological changes and/or symptoms associated with the non-rhythm pulmonary disease. A representative set of symptoms and/or physiological changes associated with non-rhythm pulmonary diseases may include, for example, dyspnea (e.g., non-specific dyspnea, orthopnea, exertional dyspnea, paroxysmal nocturnal dyspnea), abnormal concentrations of blood or respiratory gases (e.g., cyanosis, hypoxemia, hypercapnea, low pCO2, arterial acidosis, high alveolar-arterial pO2 differential), respiratory sounds (e.g., wheezing, crackles, rhonchi, fiction rub, attenuated breath sounds, snoring), pulmonary function dysfunction (e.g., low forced expiratory volume (FEV), forced vital capacity (FVC), FEV/FVC, low forced expiratory flow (FEF), high functional residual capacity (FRC), total lung capacity (TLC), high residual volume (RV), high lung compliance, slow exhalation, tachypnea, shallow breathing, high minute ventilation, respiratory failure, reduced diffusion capacity), other pulmonary conditions (e.g., hemoptysis, cough, pleuritic chest pain, local inflammation, excess mucous production, chest pain, respiratory infection, as indicated by a slightly elevated white blood count, pulmonary mucus, overinflated lungs, alveolar wall breakdown, mucosal pulmonary edema, ventilation-perfusion mismatch, subepithelial fibrosis (chronically), respiratory muscle fatigue, high small airway resistance, hoarseness), cardiovascular conditions (e.g., pulmonary hypertension, high pulmonary vascular resistance, tachycardia, circulatory collapse, pulsus paradoxicus, syncope, hypertension, S3 heart sounds, RV hypertrophy, systolic murmur), and general systemic conditions (e.g., fever, weight loss, weight gain, night sweats, peripheral edema, high hemoglobin, fatigue, joint pain, hypersomnolence.

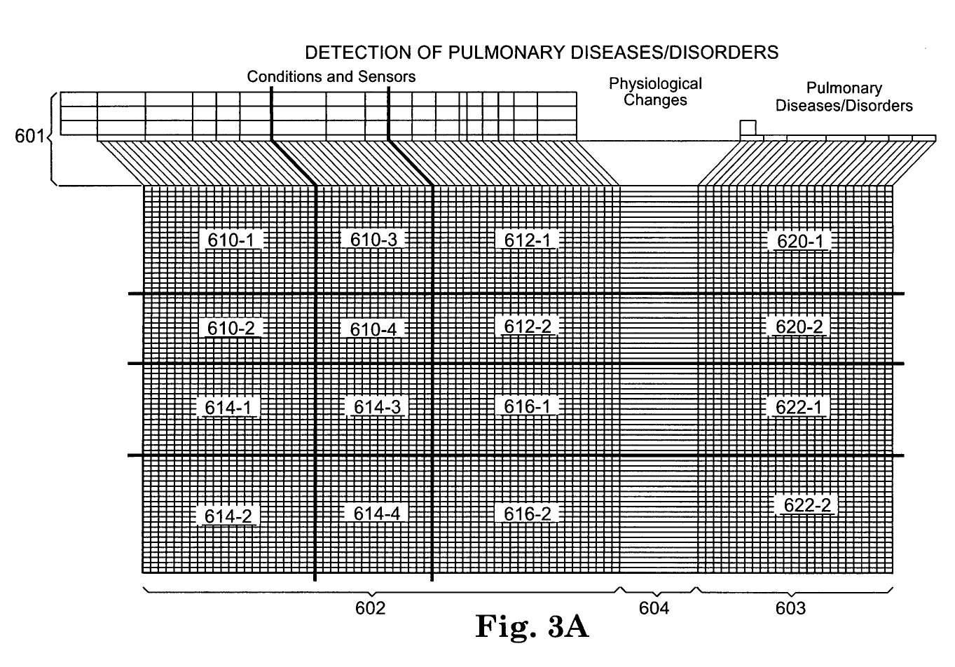

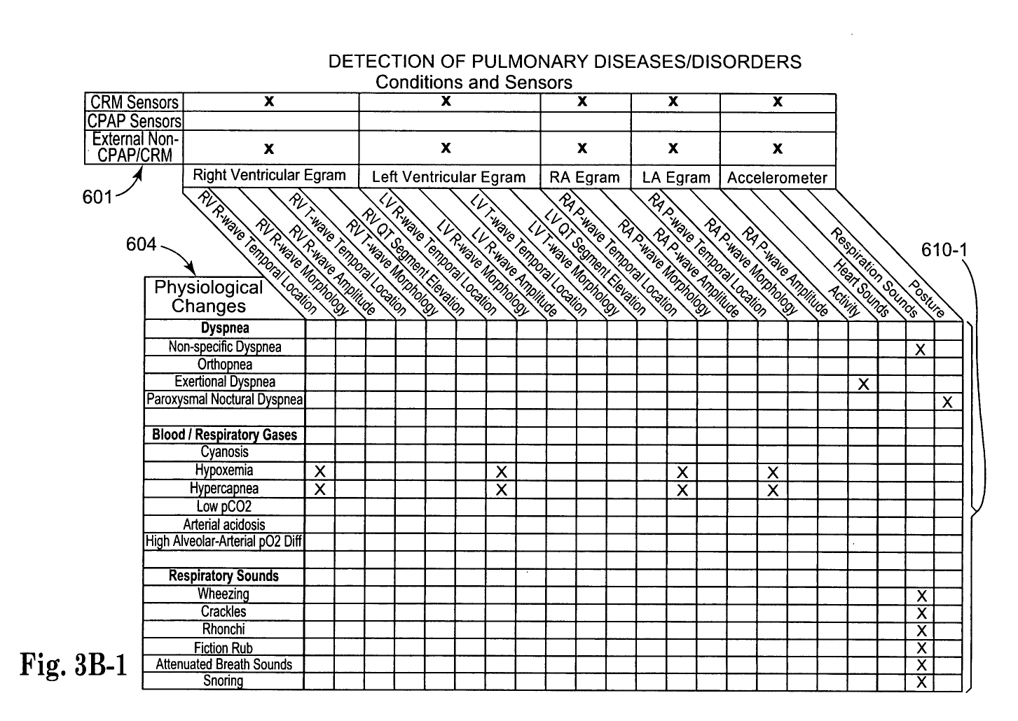

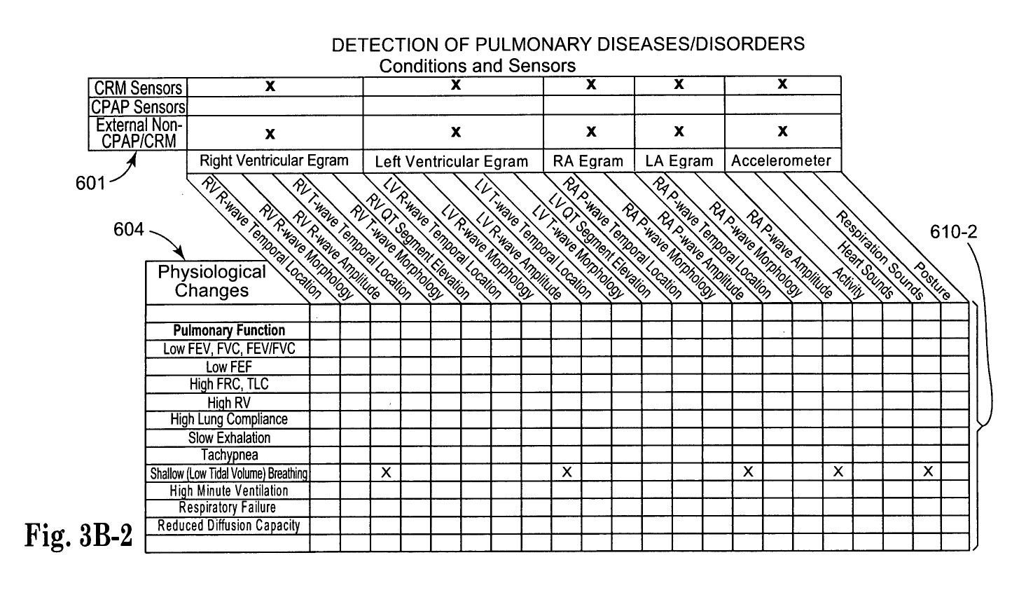

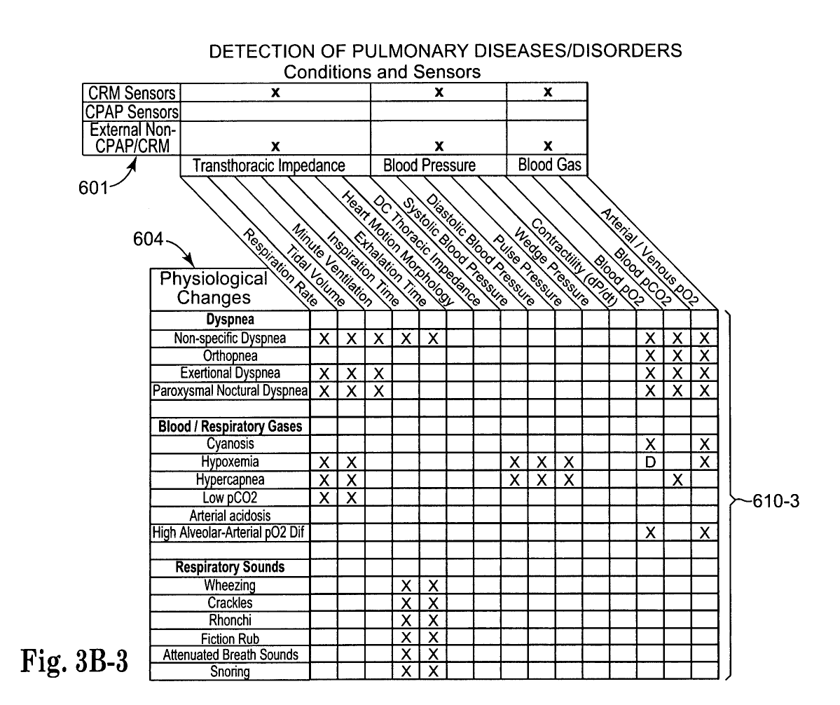

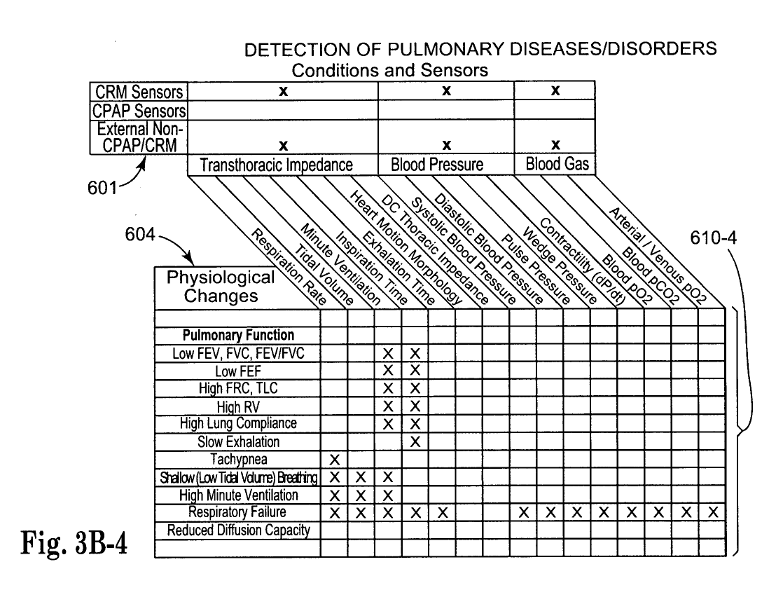

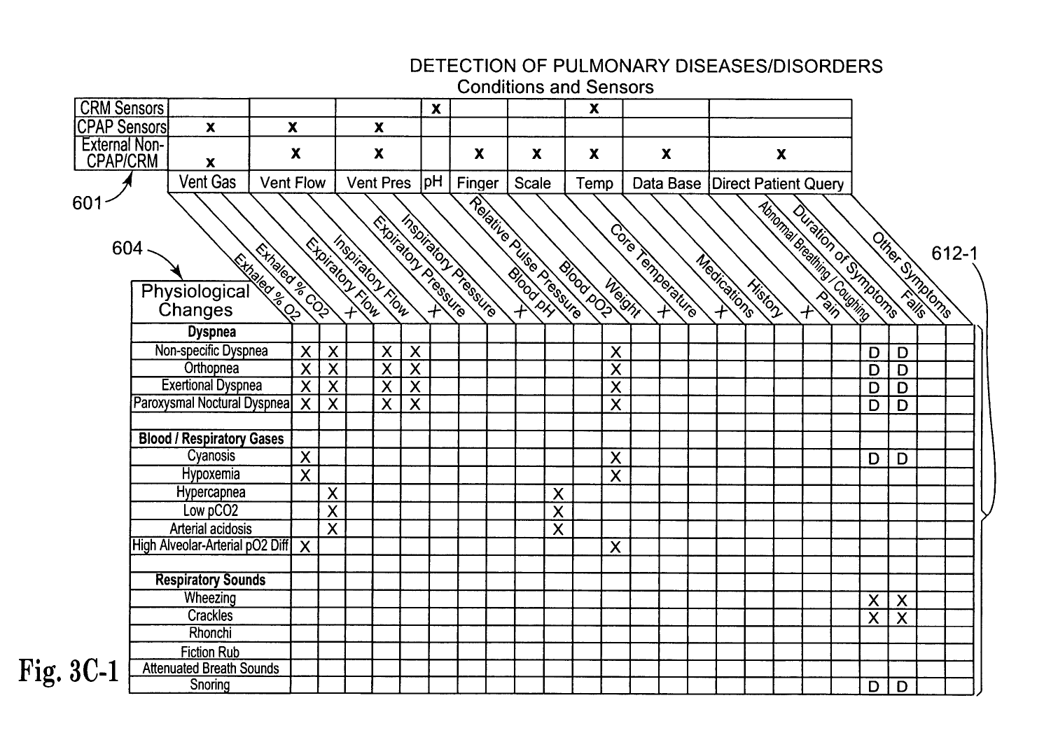

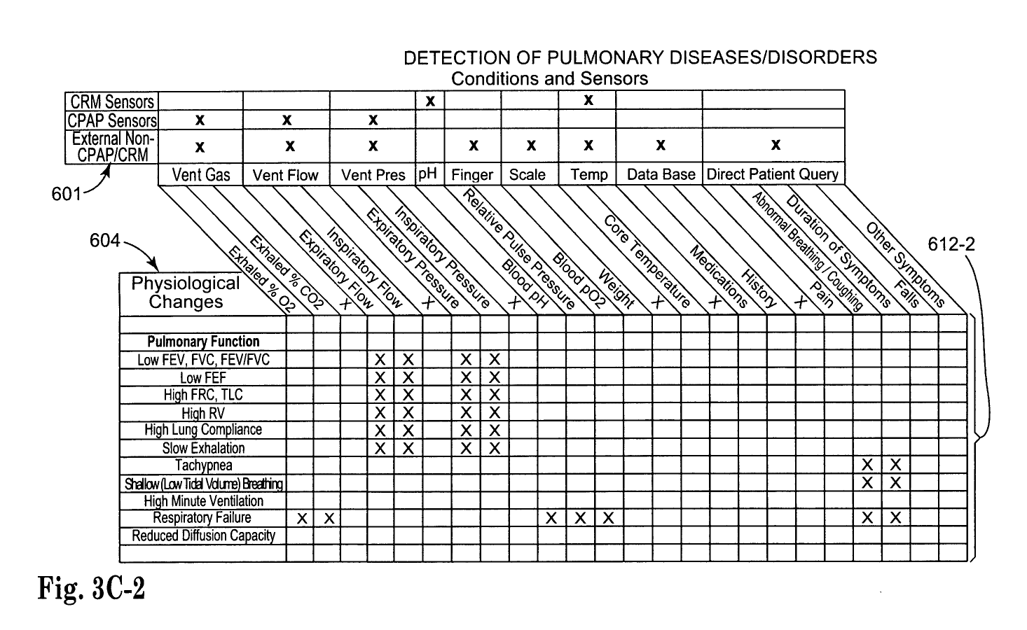

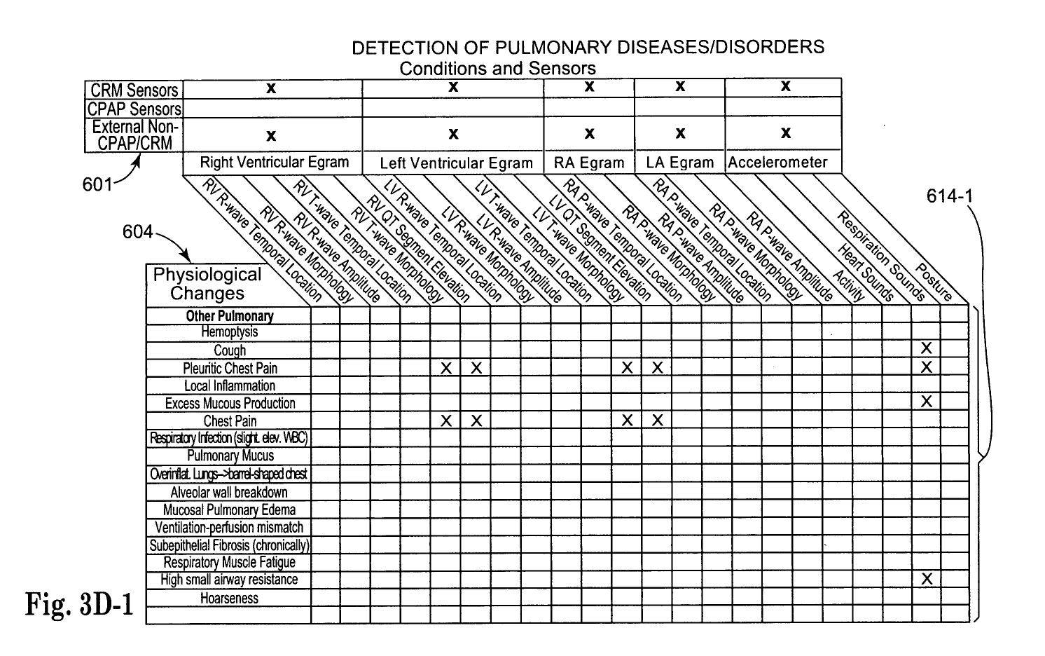

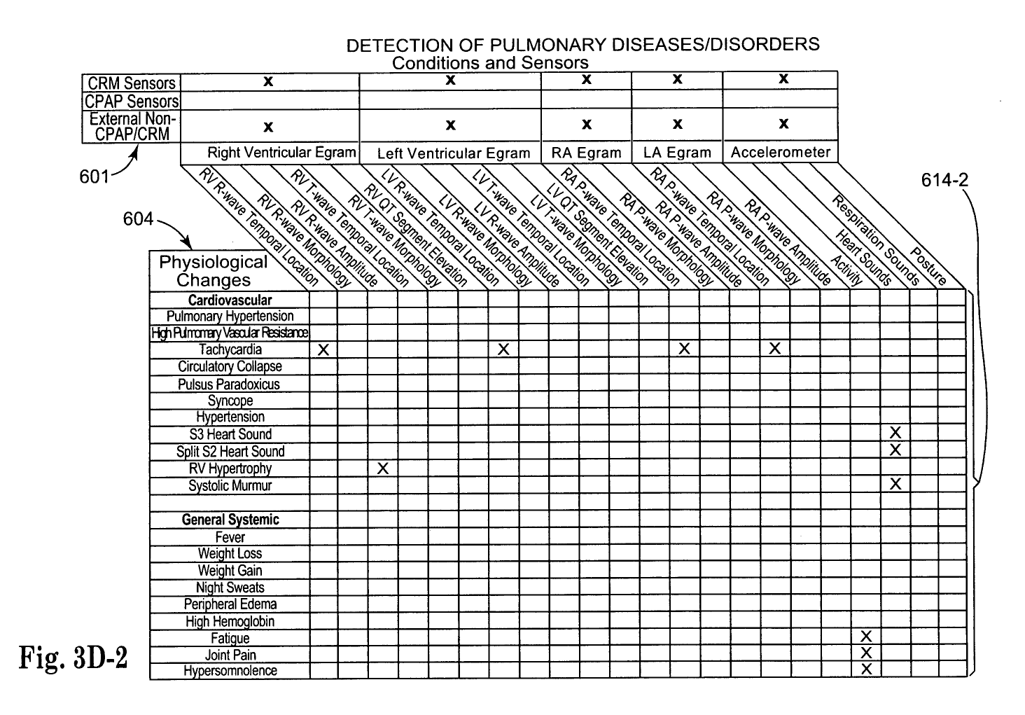

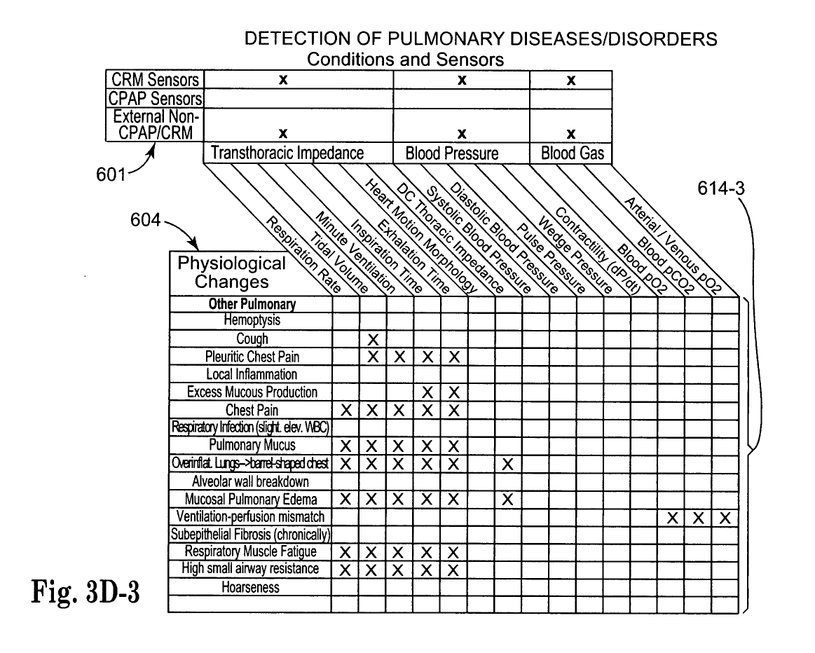

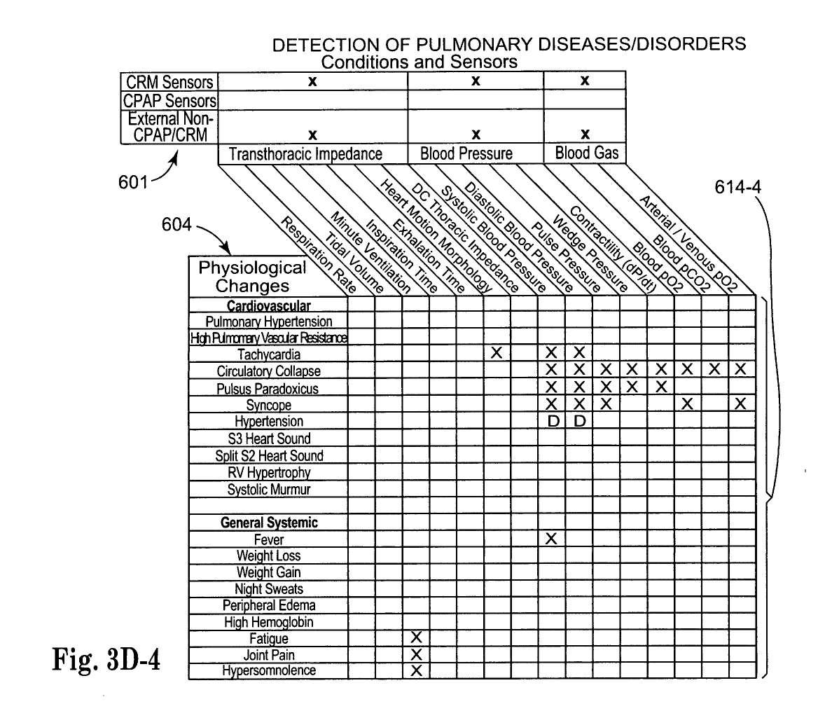

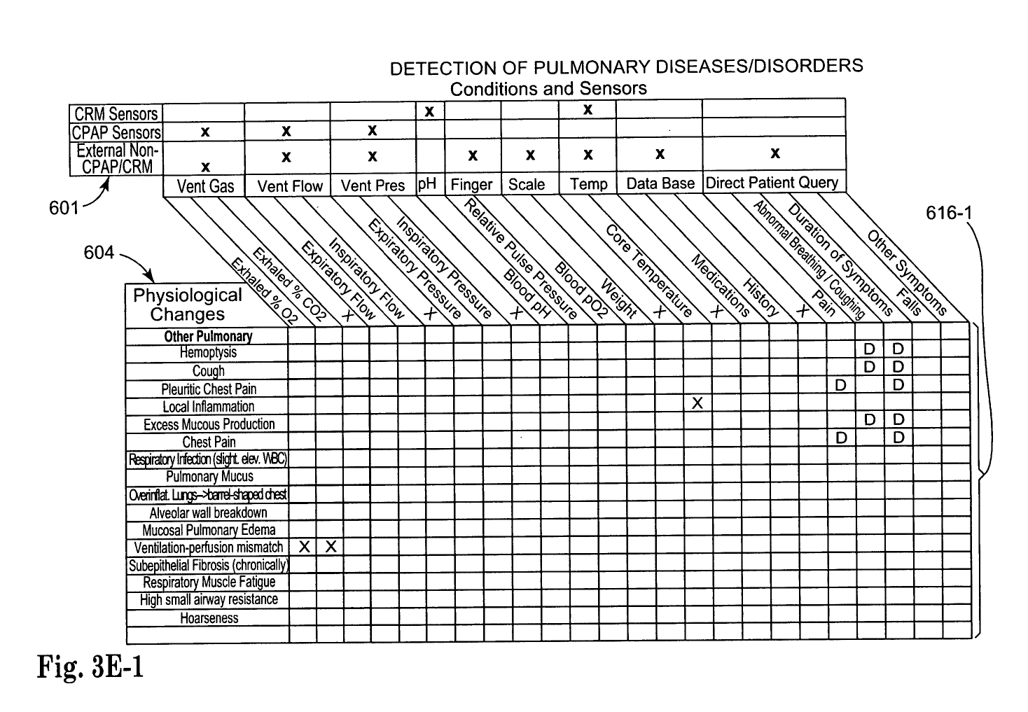

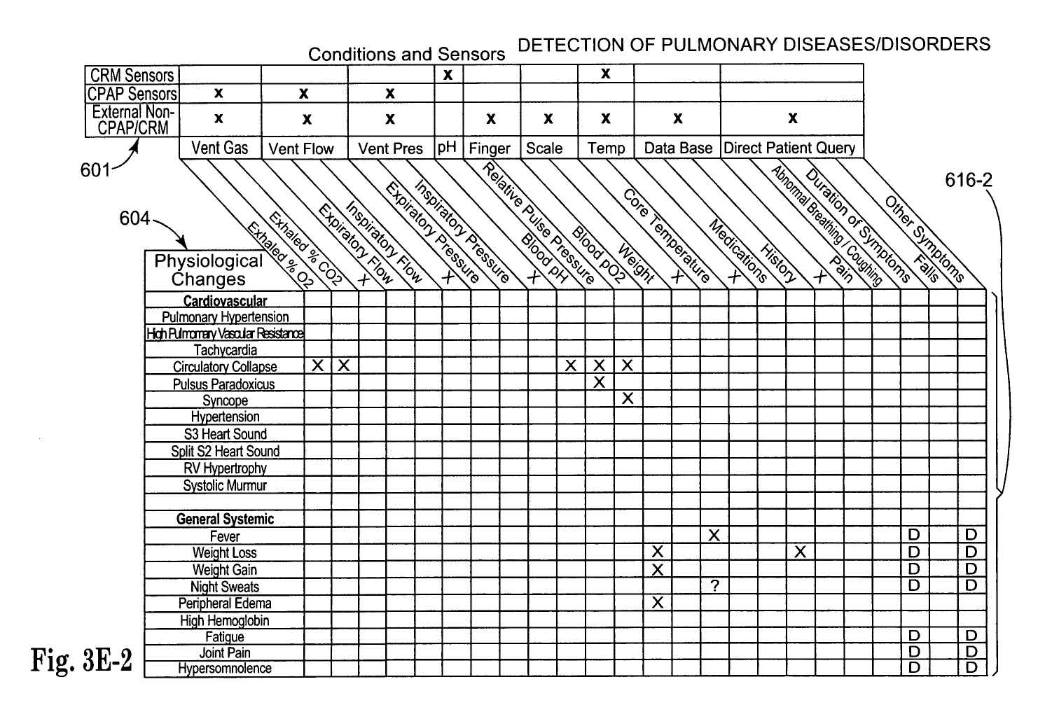

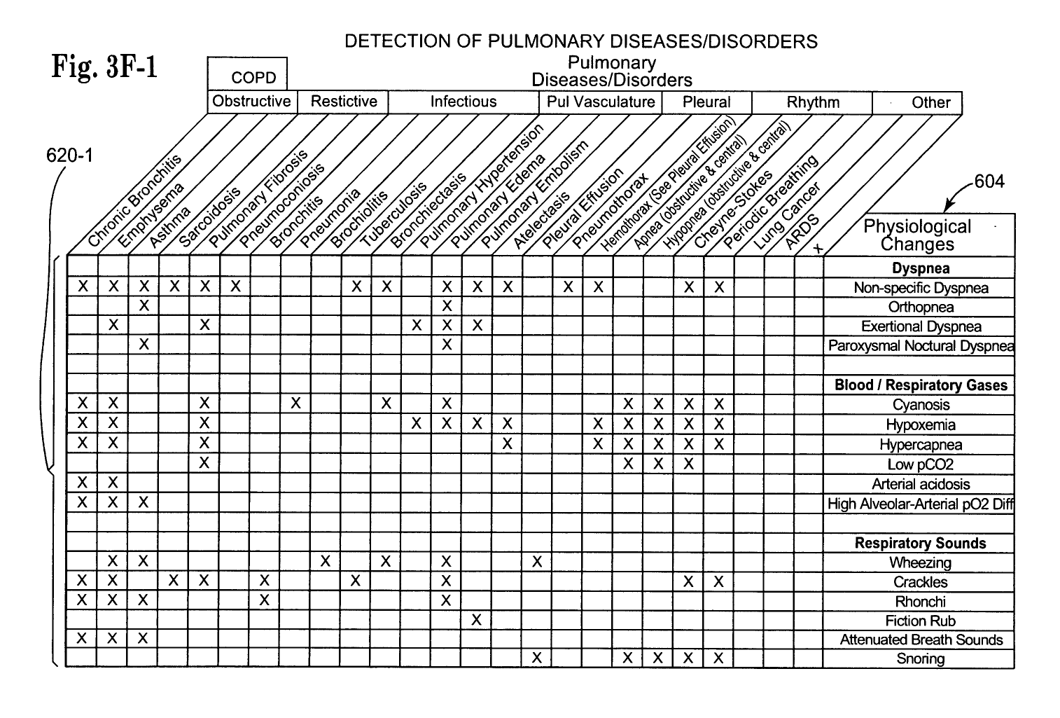

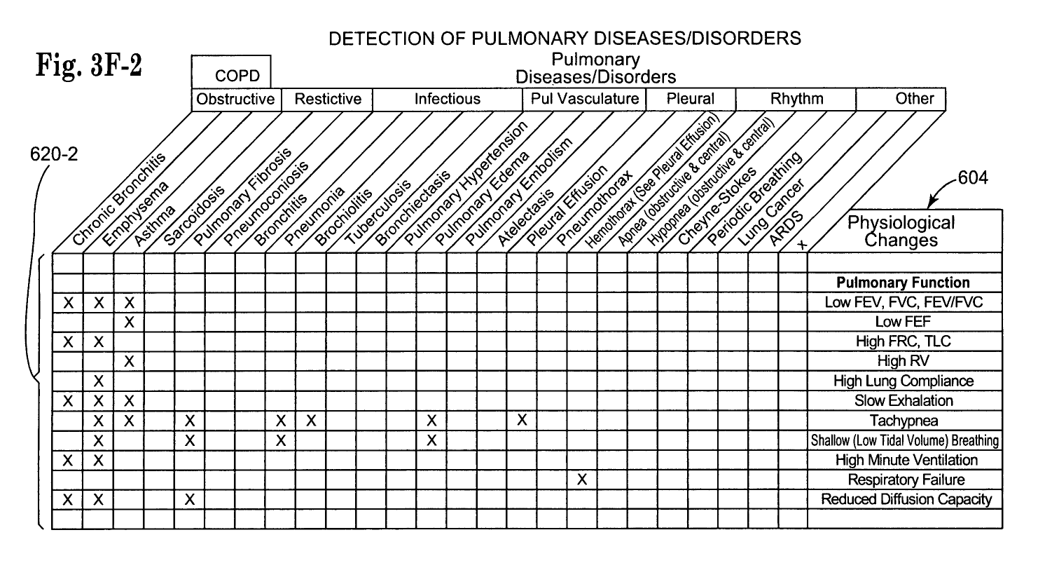

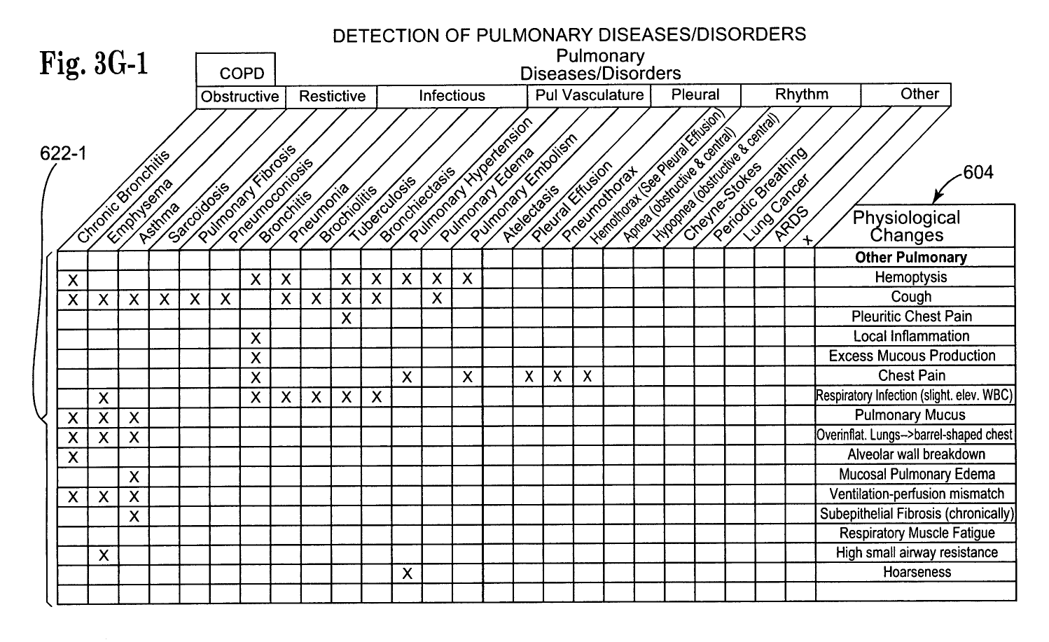

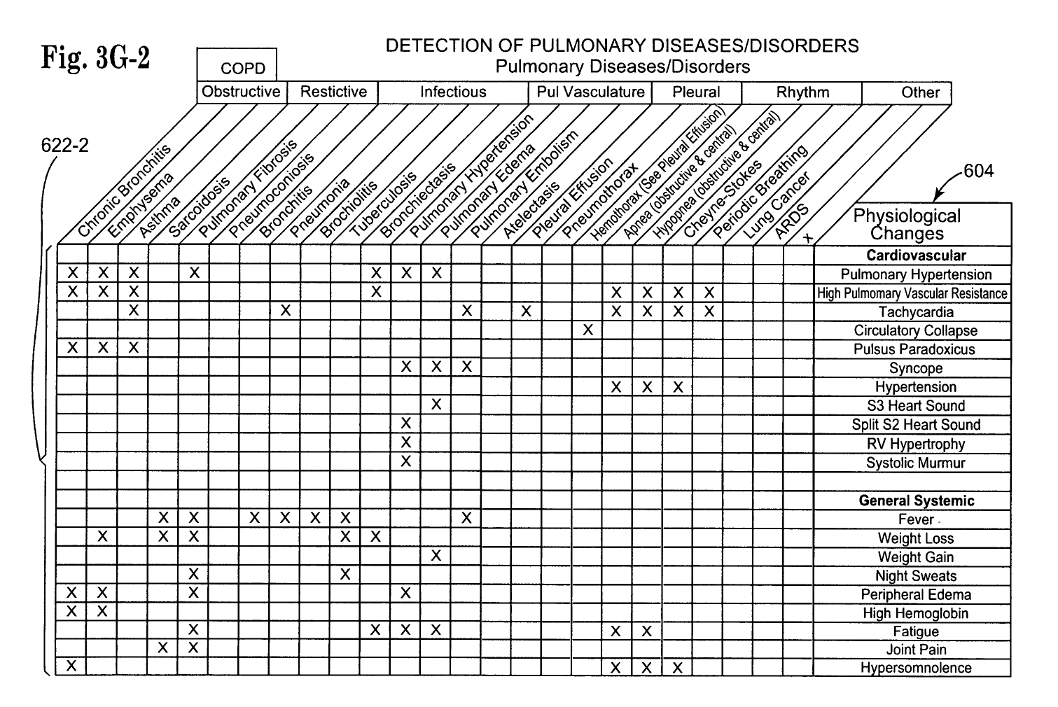

FIGS. 3A-3N, discussed in more detail below, represent a chart listing non-rhythm pulmonary disease symptoms or physiological changes, conditions indicative of the symptoms or physiological changes, and representative sensors of a respiratory therapy device, e.g., CPAP device, and a cardiac device that may be used to sense the conditions.

Referring back to FIG. 2A, the sensors and/or other input devices 235 are coupled to signal processor circuitry 230 which may be configured to energize the sensors, to receive and condition signals generated by the sensors, and/or to facilitate communication between the respiratory therapy device 210 and the sensors 235. The signal processor circuitry 230 may comprise, for example, driver circuitry, filters, sampling circuitry, A/D converter circuitry. The sensor/input device signals may be averaged, filtered, or otherwise processed by the signal processor circuitry 230 prior to use by other components of the respiratory therapy device 210.

The respiratory therapy device 210, illustrated in FIG. 2A as a positive airway pressure (xPAP) device includes a therapy control unit 220. The therapy control unit 220 comprises a flow generator 221 that pulls in air through a filter. The flow generator 221 is controlled by the pressure control circuitry 222 to deliver an appropriate air pressure to the patient. Air flows through tubing 223 coupled to the xPAP device 210 and is delivered to the patient's airway through a mask 224. In one example, the mask 224 may be a nasal mask covering only the patient's nose. In another example, the mask 224 covers the patient's nose and mouth. Other air delivery systems are also possible.

Continuous positive airway pressure (CPAP) devices deliver a set air pressure to the patient. The pressure level for the individual patient may be determined during a titration study, for example. Such a study may take place in a sleep lab, and involves determination by a sleep physician or other professional of the optimum airway pressure for the patient. The CPAP device pressure control is set to the determined level. When the patient uses the CPAP device, a substantially constant airway pressure level is maintained by the device. The constant air pressure acts a pneumatic splint to keep soft tissue in the patient's throat from collapsing and obstructing the airway.

Autotitration PAP devices are similar to CPAP devices, however, the pressure controller for autotitration devices automatically determines the air pressure delivered to the patient. Instead of maintaining a constant pressure, the autotitration PAP device evaluates sensor signals and the changing needs of the patient to deliver a variable positive airway pressure. Autotitration PAP and CPAP are often used to treat sleep disordered breathing, for example.

Bi-level positive airway pressure (bi-PAP) devices provide two levels of positive airway pressure. A higher pressure is maintained while the patient inhales. The device switches to a lower pressure during expiration. Bi-PAP devices are used to treat a variety of respiratory dysfunctions, including chronic obstructive pulmonary disease (COPD), respiratory insufficiency, and ALS or Lou Gehrig's disease, among others.

Some positive airway pressure devices may also be configured to provide both positive and negative pressure, such that negative pressure is selectively used (and de-activated) when necessary, such as when treating Cheyne-Stokes breathing, for example. The term xPAP will be used herein as a generic term for any such device, including devices using forms of positive airway pressure (and negative pressure when necessary), whether continuous or otherwise.

In accordance with various embodiments of the invention, the xPAP device 210 may include a diagnostics unit 260. The diagnostics unit 260 evaluates patient conditions sensed or input directly by the sensors/input devices 235 or derived from the sensor signals to assess a presence of a non-rhythm pulmonary disease.

In some embodiments, the therapy control unit 220 of the respiratory therapy unit 210 includes circuitry for drug therapy control 293. The drug therapy controller 293 generates a control signal to initiate, terminate, or modify drug therapy based on the assessment of the non-rhythm pulmonary disease. In one embodiment, the drug therapy comprises a gas that is delivered to the patient through the xPAP tubing 223 and mask 224. A gas therapy delivery unit is incorporated within the xPAP device 210. The drug therapy controller 293 generates a signal that controls and modulates the release of a gas by the gas therapy delivery unit 227. Various methods and systems for controlling gas therapy delivered to a patient, aspects of which may be utilized in connection with the embodiments discussed herein, are described in commonly owned U.S. Patent Application, identified by Attorney Docket No.: GUID.135PA, entitled “Methods and Systems for Control of Gas Therapy,” filed Aug. 30, 2004, and incorporated herein by reference.

The xPAP device 210 may include a communications unit 240 for communicating with one or more separate devices 270, such as a device programmer, APM system, and/or other patient-external or patient-internal monitoring, diagnostic and/or therapeutic devices. Communication between cooperating devices allows the xPAP device 210 to provide or obtain information to/from the cooperating devices or to control therapy delivered by the cooperating devices, for example.

In one implementation, illustrated in FIG. 2B, one or both of the diagnostics unit 260 and the drug therapy controller 293 may be positioned remotely with respect to the patient-external respiratory therapy device 210. The xPAP device 210 may include a monitoring unit 250 including a memory for storing data related to the non-rhythm pulmonary disease or other data. In one scenario, monitoring unit 250 may sense the one or more patient conditions and may store data related to the sensed conditions. The monitoring unit may collect and store data hourly, nightly, weekly, randomly or according to a time schedule that corresponds to the patient's usage times of the respiratory therapy device 210. Typically an xPAP device is used nightly for treatment of sleep apnea and/or other breathing rhythm disorders. The xPAP device 210 may collect data from the sensors/input devices 235 during one or more periods of time that the device is used. The presence of the non-rhythm pulmonary disease may be assessed based on the collected data. Assessment of the non-rhythm pulmonary disease may involve assessment of the onset, progression, regression and/or offset of the disease.

In the implementation illustrated in FIG. 2B, the respiratory therapy device 210 may transmit information about conditions sensed by the respiratory therapy device 210 to the diagnosis unit 260 of a remotely located device 270. The diagnosis unit 260 assesses the non-rhythm pulmonary disease presence based on the transmitted information. The drug therapy controller develops a control signal for controlling drug therapy delivery. The remotely located device 270 transmits the control signal to a drug delivery unit. In one embodiment, the drug delivery unit may be activated to release a gas, e.g., albuterol, into the airflow of the respiratory therapy device. In other embodiments, other types of drug delivery methodologies, such as a drug pump, an electrically activated drug patch, and/or other types of drug delivery devices may be employed.

The remote device 270 may comprise a patient-external or patient-internal medical device. The remote device 270 may be configured, for example, as a cardiac diagnostic and/or therapeutic device. In one configuration, for example, the remote device 270 may comprise a cardiac rhythm management system, such as a pacemaker, defibrillator, or cardiac resynchronizer.

In some embodiments, as illustrated in FIGS. 2C and 2D, an external respiratory therapy device may be used in combination with an implantable device, such as an implantable cardiac rhythm management device, to detect and/or monitor a presence of a non-rhythm pulmonary disease. The system illustrated in FIG. 2C includes an external respiratory therapy device 210 and a cardiac device 292, such as an implantable pacemaker, defibrillator, cardioverter, cardiac resynchronizer or cardiac monitor. Both the respiratory therapy device 210 and the cardiac device 292 are equipped with sensors/input devices 235, 236 for sensing conditions associated with symptoms of one or more non-rhythm pulmonary diseases. For example, each of the respiratory therapy device 210 and the cardiac device 292 may sense a subset of the conditions listed in FIGS. 3A-3G.

The respiratory therapy device 210 may transmit its sensed condition information to the cardiac device 292, e.g., over a wireless communications link. The cardiac device 292 includes a diagnostics unit 260 configured to assess a presence of one or more non-rhythm pulmonary diseases by evaluating the conditions sensed by the respiratory device and/or by evaluating additional conditions sensed by the cardiac device.

The diagnostic unit 260 may assess the one or more non-rhythm pulmonary diseases, for example, by comparing sensed conditions to corresponding sets of criteria indicative of the non-rhythm pulmonary diseases. In this system depicted in FIG. 2C, the cardiac device 292 includes a drug therapy controller 293 that develops control signals to control a drug therapy delivered to the patient. The cardiac device 292 transmits signals to the drug delivery device 295 to initiate, modify or terminate drug therapy delivered to the patient based on the assessment of the pulmonary disease.

In an alternate implementation one or both of the diagnostics processor 260 and the drug therapy controller 293 may be disposed in the respiratory therapy device housing.

The block diagram of FIG. 2D illustrates another arrangement of a pulmonary disease assessment and drug therapy delivery system. In this example, the system includes a respiratory therapy device 210 and a cardiac device 292. The respiratory therapy device 210 and the cardiac device 292 communicate with a remote diagnostic unit 260, such as may be incorporated in an APM system 230. The respiratory therapy device 210 and the cardiac device 292 are each equipped with sensors/input devices 236, 237 for sensing conditions associated with one or more non-rhythm pulmonary diseases. The respiratory therapy device 210 and the cardiac device 292 may transmit sensed condition information to the diagnostic unit 260 through a wireless or wired communication links. The pulmonary disease diagnostic unit 260 is configured to use the information transmitted by the respiration therapy device 210 and the cardiac device 292 to assess the presence of one or more non-rhythm pulmonary diseases.

A drug therapy controller 293 uses the assessment of the non-rhythm pulmonary disease to develop signals from controlling the drug therapy delivered to the patient. In one configuration, the diagnostic unit 260 and the drug therapy controller 293 may be configured as components of an APM system 230. A control signal developed by the drug therapy controller may be used to activate, modify, terminate or otherwise control therapy delivered by a drug therapy device 295.

Assessment of conditions indicative of non-rhythm pulmonary diseases/disorders may include assessing the patient's pulmonary function as previously described. The charts provided in FIGS. 3A-3G illustrate conditions and sensors that may be used to determine physiological changes associated with various non-rhythm pulmonary diseases and disorders. The charts depicted in FIGS. 3A-3G illustrate relationships between various physiological changes and/or disease symptoms associated with non-rhythm pulmonary diseases. FIG. 3A lists representative sets of non-rhythm pulmonary diseases that may be assessed in accordance with embodiments of the invention. The representative set of non-rhythm pulmonary diseases that may be assessed includes, for example, obstructive pulmonary diseases (e.g., chronic bronchitis, emphysema, asthma), restrictive pulmonary diseases (e.g., sarcoidosis, pulmonary fibrosis, pneumoconiosis), infections pulmonary diseases (e.g., bronchitis, pneumonia, bronchiolitis, tuberculosis, and bronchiectasis), pulmonary vasculature diseases (e.g., pulmonary hypertension, pulmonary edema, pulmonary embolism, atalectasis), and diseases of the pleural cavity (e.g., pleural effusion, pneumothorax, and hemothorax).

The non-rhythm pulmonary diseases listed in FIG. 3A are cross-referenced with the physiological changes and/or symptoms associated with the non-rhythm pulmonary disease. The physiological changes and/or symptoms are cross referenced with conditions indicative of the physiological changes and/or symptoms. Sensors used to sense the conditions indicative of the physiological changes or symptoms are provided in FIG. 3A. Sensors of the respiratory therapy device may include, for example, ventilation gas, ventilation flow and/or ventilation pressure sensors, or other sensors for example.

The left section 602 of FIG. 3A illustrates various conditions that may be sensed using sensors of a respiratory therapy device (CPAP), a cardiac device (CRM), or an external non-CPAP, non-CRM device. The top section 601 lists various conditions that may be sensed and information about sensors used to sense the conditions. The center section 604 of FIG. 3A provides physiological changes and/or symptoms that may be evaluated using the conditions listed in the left section 602. The right section 603 of FIG. 3A provides pulmonary diseases/disorders. The presence of the pulmonary diseases/disorders of the right section 603 may be assessed based on the physiological changes and/or symptoms of the center section 604.

For legibility, the left and right sections 602, 603 of FIG. 3A are divided into six portions, FIGS. 3B-3G. FIG. 3B represents the upper left portion 610 of the left section 602 of FIG. 3A. FIG. 3C represents the upper right portion 612 of the left section 602 of FIG. 3A. FIG. 3D represents the lower left portion 614 of the left section 602 of FIG. 3A. FIG. 3E represents the lower right portion 616 of the left section 602 of FIG. 3A. FIG. 3F represents the upper portion 620 of the right section 603 of FIG. 3A. FIG. 3G represents the lower portion 622 of the right section 603 of FIG. 3A. Relevant portions of the center section 604 and the top section 601 of FIG. 3A appear in each of the FIGS. 3B-3G for convenience.

An example of how FIGS. 3A-3G may be used follows. Referring to FIGS. 3F and 3G, the restrictive pulmonary disorder pneumoconiosis produces the physiological changes non-specific dyspnea (FIG. 3F) and cough (FIG. 3G). Non-specific dyspnea (FIG. 3F) and cough (FIG. 3G) are indicated by marks in the column denoted pneumoconiosis in FIGS. 3F and 3G, respectively. Non-specific dyspnea may be detected based on one or more of the conditions listed in the row for non-specific dyspnea illustrated in FIGS. 3B and 3D. The conditions include duration of symptoms, abnormal breathing/coughing , blood pO2, inspiratory flow, expiratory flow, exhaled % CO2 and exhaled % O2, illustrated in FIG. 3D. The conditions also include arterial/venous pO2, blood pCO2, blood pO2, exhalation time, inspiration time, minute ventilation, tidal volume, respiration rate, and/or respiration sounds illustrated in FIG. 3B.

A cardiac device, e.g., a cardiac rhythm management system or other implantable cardiac device may include egram electrodes inserted into one or more chambers of the heart. The egram electrodes may be used to sense various cardiac conditions, including right ventricular, left ventricular, right atrial, and left atrial cardiac conditions. An illustrative list of conditions that may be sensed using the egram sensors may include, right ventricular (RV) R-wave temporal location, RV R-wave morphology, RV-R-wave amplitude, RV-T-wave morphology, RV-QT segment elevation, left ventricular (LV) R-wave temporal location, LV R-wave morphology, LV-R-wave amplitude, LV-T-wave morphology, LV-QT segment, right atrial (RA) P-wave temporal location, RA P-wave morphology, RA P-wave amplitude, LA P-wave temporal location, LA P-wave morphology, and LA P-wave amplitude.

Implantable cardiac rhythm management systems may include respiration and/or movement-based activity sensors for adjusting the pacing rate to accommodate the patient's level of activity. An accelerometer incorporated in an implantable cardiac device may be used to sense patient activity. The accelerometer may also be used to sense heart sounds, respiration sounds and/or posture, among other conditions.

The respiration-based activity sensor of an implantable cardiac device may involve sensing the patient's transthoracic impedance to determine a respiration rate. The signal from the transthoracic impedance sensor may additionally or alternatively be used to determine conditions such as tidal volume, minute ventilation, inspiration time, exhalation time, heart beat motion morphology, DC transthoracic impedance, among other conditions.

A pressure sensor of the cardiac device may be used to detect systolic and/or diastolic blood pressure, pulse pressure, wedge pressure and/or contractility dP/dt. A blood gas sensor of the cardiac device may be used to sense blood pCO2, blood pO2, arterial or venous pO2. The cardiac device may further include a blood pH sensor and a temperature sensor.

The presence of a disorder/disease, such as those listed in FIGS. 3A-3G, may be assessed by based on physiological changes and/or symptoms associated with the disorder/disease. The physiological changes and/or symptoms may be detected using conditions sensed by a sensor system of a respiratory therapy device alone or in combination with the sensor systems of other therapeutic or diagnostic medical devices, such as a pacemaker or cardiac rhythm management system. If the sensed conditions indicate that the physiological changes or symptoms of a disease or disorder are consistent with a threshold level, the presence of the disease or disorder may be determined.

Assessment of disease presence may be based on relative changes in one or more conditions indicative of physiological changes or symptoms caused by the disease. For example, assessment of a presence of a disease or disorder may be accomplished by evaluating the changes in conditions indicative of physiological changes or symptoms caused by the disease. The changes in the one or more conditions may be compared to threshold criteria. If changes in the conditions indicative of physiological changes or symptoms caused by the disease are consistent with threshold levels, a presence of the disease or disorder may be determined.

In a further example, the threshold criteria may involve relationships between the conditions indicative of physiological changes or symptoms caused by the disease. The presence of a disease may be assessed by evaluating relationships between conditions indicative of physiological changes or symptoms caused by the disease. For example, assessment of a disease may involve the determination that levels or amounts of two or more conditions have a certain relationship with one another. If relationships between the conditions indicative of physiological changes or symptoms caused by the disease are consistent with threshold relationship criteria, the disease or disorder may be present.

The results of pulmonary function testing, along with other physiological conditions measured by the CPAP and/or other devices of the system, may be compared to initial or baseline results to detect changes and/or determine trends in the patient's cardiopulmonary status over time. The changes from baseline values may be used to discern a presence of disease processes. Further, over time, a database of information about relevant conditions and specific to the patient is established. The information may be used to develop sets of criteria specific to the patient and associated with the presence of a particular cardiac and/or pulmonary disease processes. Thus, in some implementations, the system may learn to recognize the presence of disease based on the history of symptoms and/or physiological changes that occur in a particular patient.

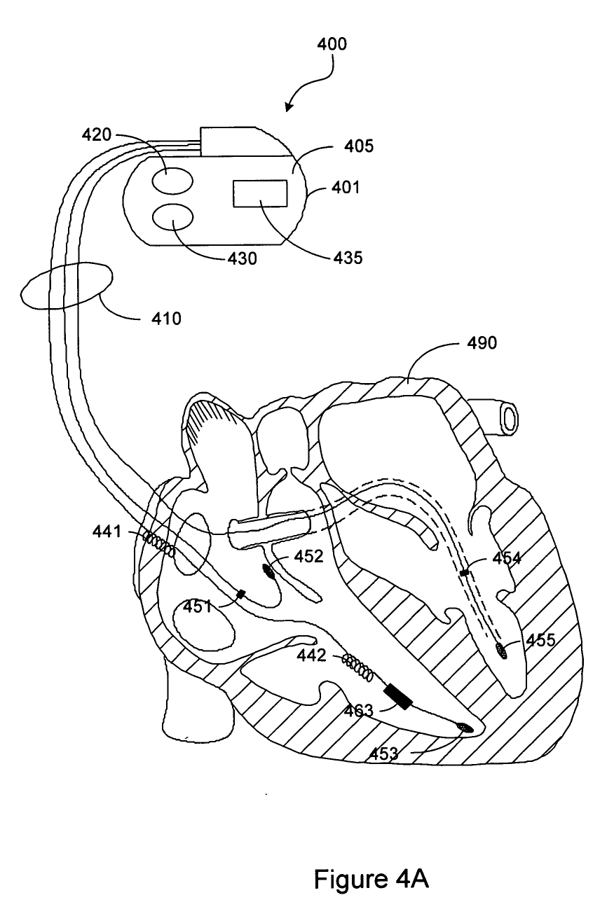

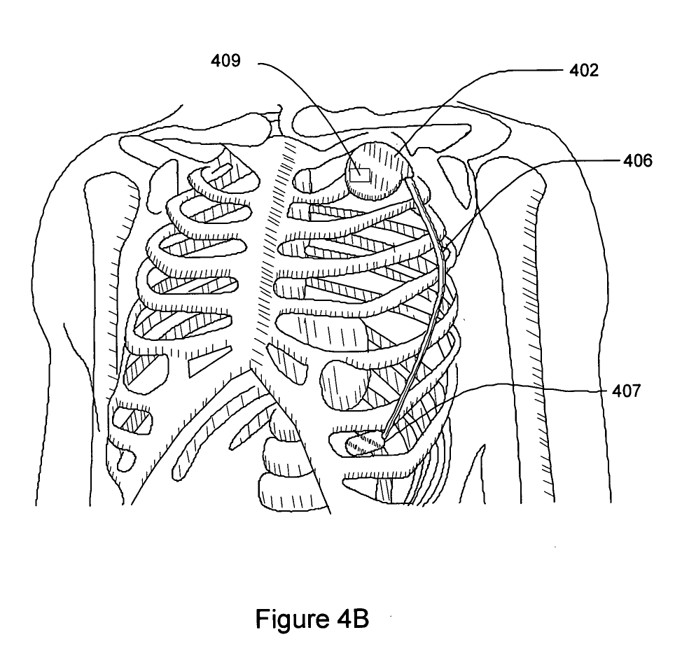

FIG. 4A is a partial view of an implantable device that may include circuitry 435 for implementing a pulmonary disease assessment and drug therapy control in accordance with embodiments of the invention. In this example, the implantable device comprises a cardiac rhythm management device (CRM) 400 including an implantable pulse generator 405 electrically and physically coupled to an intracardiac lead system 410. Circuitry for implementing a system providing non-rhythm pulmonary disease assessment and drug delivery may alternatively be implemented in a variety of implantable monitoring, diagnostic, and/or therapeutic devices, such as an implantable cardiac monitoring device, an implantable drug delivery device, or an implantable neurostimulation device, for example.

Portions of the intracardiac lead system 410 are inserted into the patient's heart 490. The intracardiac lead system 410 includes one or more electrodes configured to sense electrical cardiac activity of the heart, deliver electrical stimulation to the heart, sense the patient's transthoracic impedance, and/or sense other physiological parameters, e.g., cardiac chamber pressure or temperature. Portions of the housing 401 of the pulse generator 405 may optionally serve as a can electrode.

Communications circuitry is disposed within the housing 401 for facilitating communication between the pulse generator 405 and an external communication device, such as a portable or bed-side communication station, patient-carried/worn communication station, or external programmer, for example. The communications circuitry can also facilitate unidirectional or bidirectional communication with one or more implanted, external, cutaneous, or subcutaneous physiologic or non-physiologic sensors, patient-input devices and/or information systems.

The pulse generator 405 may optionally incorporate a motion sensor 420 that may be implemented as an accelerometer positioned in or on the housing 401 of the pulse generator 405. The motion sensor 420 may be optionally configured to sense activity level, respiration sounds (e.g., rales, coughing), heart sounds (e.g., S1-S4 heart sounds, murmurs), and/or chest wall movements associated with respiratory effort, for example.

The lead system 410 of the CRM 400 may incorporate one or more transthoracic impedance sensors that may be used to acquire the patient's respiration waveform, or other respiration-related information. The transthoracic impedance sensor may include, for example, one or more intracardiac electrodes 441, 442, 451-455, 463 positioned in one or more chambers of the heart 490. The intracardiac electrodes 441, 442, 451-455, 463 may be coupled to impedance drive/sense circuitry 430 positioned within the housing of the pulse generator 405.

In one implementation, impedance drive/sense circuitry 430 generates a current that flows through the tissue between an impedance drive electrode 451 and a can electrode on the housing 401 of the pulse generator 405. The voltage at an impedance sense electrode 452 relative to the can electrode changes as the patient's transthoracic impedance changes. The voltage signal developed between the impedance sense electrode 452 and the can electrode is detected by the impedance sense circuitry 430. Other locations and/or combinations of impedance sense and drive electrodes are also possible.

FIG. 4B is a diagram illustrating an implantable transthoracic cardiac device that may be used in connection with controlling drug therapy in accordance with embodiments of the invention. The implantable device illustrated in FIG. 4B is an implantable transthoracic cardiac sensing and/or stimulation (ITCS) device that may be implanted under the skin in the chest region of a patient. The ITCS device may, for example, be implanted subcutaneously such that all or selected elements of the device are positioned on the patient's front, back, side, or other body locations suitable for sensing cardiac activity and delivering cardiac stimulation therapy. It is understood that elements of the ITCS device may be located at several different body locations, such as in the chest, abdominal, or subclavian region with electrode elements respectively positioned at different regions near, around, in, or on the heart.

Circuitry for implementing drug therapy control may be positioned within the primary housing of the ITCS device. The primary housing (e.g., the active or non-active can) of the ITCS device, for example, may be configured for positioning outside of the rib cage at an intercostal or subcostal location, within the abdomen, or in the upper chest region (e.g., subclavian location, such as above the third rib). In one implementation, one or more electrodes may be located on the primary housing and/or at other locations about, but not in direct contact with the heart, great vessel or coronary vasculature.

In another implementation, one or more electrodes may be located in direct contact with the heart, great vessel or coronary vasculature, such as via one or more leads implanted by use of conventional transvenous delivery approaches. In another implementation, for example, one or more subcutaneous electrode subsystems or electrode arrays may be used to sense cardiac activity and deliver cardiac stimulation energy in an ITCS device configuration employing an active can or a configuration employing a non-active can. Electrodes may be situated at anterior and/or posterior locations relative to the heart.

In the configuration shown in FIG. 4B, a subcutaneous electrode assembly 407 can be positioned under the skin in the chest region and situated distal from the housing 402. The subcutaneous and, if applicable, housing electrode(s) can be positioned about the heart at various locations and orientations, such as at various anterior and/or posterior locations relative to the heart. The subcutaneous electrode assembly 407 is coupled to circuitry within the housing 402 via a lead assembly 406. One or more conductors (e.g., coils or cables) are provided within the lead assembly 406 and electrically couple the subcutaneous electrode assembly 407 with circuitry in the housing 402. One or more sense, sense/pace or defibrillation electrodes can be situated on the elongated structure of the electrode support, the housing 402, and/or the distal electrode assembly (shown as subcutaneous electrode assembly 407 in FIG. 4B).

It is noted that the electrode and the lead assemblies 407, 406 can be configured to assume a variety of shapes. For example, the lead assembly 406 can have a wedge, chevron, flattened oval, or a ribbon shape, and the subcutaneous electrode assembly 407 can comprise a number of spaced electrodes, such as an array or band of electrodes. Moreover, two or more subcutaneous electrode assemblies 407 can be mounted to multiple electrode support assemblies 406 to achieve a desired spaced relationship amongst subcutaneous electrode assemblies 407.

In particular configurations, the ITCS device may perform functions traditionally performed by cardiac rhythm management devices, such as providing various cardiac monitoring, pacing and/or cardioversion/defibrillation functions. Exemplary pacemaker circuitry, structures and functionality, aspects of which can be incorporated in an ITCS device of a type that may benefit from multi-parameter sensing configurations, are disclosed in commonly owned U.S. Pat. Nos. 4,562,841; 5,284,136; 5,376,476; 5,036,849; 5,540,727; 5,836,987; 6,044,298; and 6,055,454, which are hereby incorporated herein by reference in their respective entireties. It is understood that ITCS device configurations can provide for non-physiologic pacing support in addition to, or to the exclusion of, bradycardia and/or anti-tachycardia pacing therapies. Exemplary cardiac monitoring circuitry, structures and functionality, aspects of which can be incorporated in an ITCS of the present invention, are disclosed in commonly owned U.S. Pat. Nos. 5,313,953; 5,388,578; and 5,411,031, which are hereby incorporated herein by reference in their respective entireties.

An ITCS device can incorporate circuitry, structures and functionality of the subcutaneous implantable medical devices disclosed in commonly owned U.S. Pat. Nos. 5,203,348; 5,230,337; 5,360,442; 5,366,496; 5,397,342; 5,391,200; 5,545,202; 5,603,732; and 5,916,243 and commonly owned U.S. patent application Ser. No. 60/462,272, filed Apr. 11, 2003, Ser. No. 10/462,001, filed Jun. 13, 2003, Ser. No. 10/465,520, filed Jun. 19, 2003, Ser. No. 10/820,642 filed Apr. 8, 2004 and Ser. No. 10/821,248, filed Apr. 8, 2004 which are incorporated herein by reference.

The housing of the ITCS device may incorporate components 409 of pulmonary disease assessment unit and/or a drug therapy controller. In one embodiment, the housing of the ITCS device includes a diagnostics unit. The diagnostics unit may be coupled to one or more sensors, patient input devices, and/or information systems as described herein. In some embodiments, the housing of the ITCS device may incorporate components of a drug therapy controller. The drug therapy controller may be coupled through wire leads or wirelessly to a drug delivery device. In other embodiments, the ITCS housing may incorporate both diagnostics circuitry and drug therapy controller circuitry.

In one implementation, the ITCS device may include an impedance sensor configured to sense the patient's transthoracic impedance. The impedance sensor may include the impedance drive/sense circuitry incorporated with the housing 402 of the ITCS device and coupled to impedance electrodes positioned on the can or at other locations of the ITCS device, such as on the subcutaneous electrode assembly 407 and/or lead assembly 406. In one configuration, the impedance drive circuitry generates a current that flows between a subcutaneous impedance drive electrode and a can electrode on the primary housing of the ITCS device. The voltage at a subcutaneous impedance sense electrode relative to the can electrode changes as the patient's transthoracic impedance changes. The voltage signal developed between the impedance sense electrode and the can electrode is sensed by the impedance drive/sense circuitry.

Communications circuitry is disposed within the housing 402 for facilitating communication between the ITCS device, including the monitoring unit 409, and an external communication device, such as a portable or bed-side communication station, patient-carried/worn communication station, or external programmer, for example. The communications circuitry can also facilitate unidirectional or bidirectional communication with one or more external, cutaneous, or subcutaneous physiologic or non-physiologic sensors.

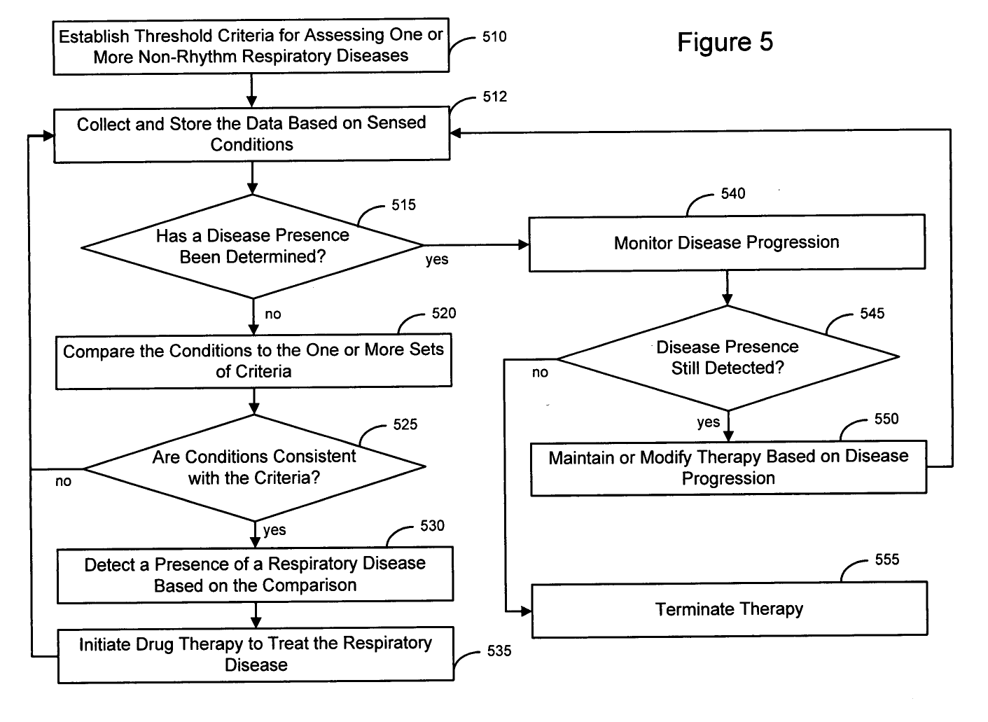

FIG. 5 is a flowchart illustrating a method in accordance with embodiments of the invention. Criteria sets for assessment of the non-rhythm pulmonary diseases are established 510. A respiratory therapy device such as a CPAP device is used to sense conditions modulated by disease symptoms. The sensor information may be collected 512 periodically, e.g., nightly, and stored for evaluation. If a presence of the disease was not previously determined 515, then the levels of the sensed conditions are compared 520 to a set of criteria associated with the disease. If levels of the conditions are consistent 525 with the threshold criteria levels, then a presence of the disease is determined 530. Drug therapy is initiated 535 to treat the respiratory disease.

If levels of the conditions are not consistent 525 with the threshold criteria levels, then the system continues to sense conditions modulated by disease symptoms and collect 512 and store data based on the sensed conditions.

If the presence of the disease was previously determined 515, then the progression of the disease may be monitored 540 based on the conditions and/or criteria used to determine a presence of the disease, or using other conditions and/or criteria. If the disease presence is still detected 545 based on the conditions and criteria used for monitoring, then therapy is maintained or modified 550 based on the disease progression. Disease progression may be determined, for example, by trending one or more conditions used for monitoring the disease presence over a period of time. Modifications to the drug therapy may be made based on the condition trends. If the disease presence is no longer detected 545, then the drug therapy may be terminated 555.

Methods, devices, and systems in accordance with the present invention may incorporate one or more of the features, structures, methods, or combinations thereof described herein. For example, a medical system may be implemented to include one or more of the features and/or processes described herein. It is intended that such a method, device, or system need not include all of the features and functions described, but may be implemented to include one or more selected features and functions that provide unique structures and/or functionality.

A number of the examples presented herein involve block diagrams illustrating functional blocks used for monitoring functions in accordance with embodiments of the present invention. It will be understood by those skilled in the art that there exist many possible configurations in which these functional blocks can be arranged and implemented. The examples depicted herein provide examples of possible functional arrangements used to implement the approaches of the invention.

The components and functionality depicted as separate or discrete blocks/elements in the figures in general can be implemented in combination with other components and functionality. The depiction of such components and functionality in individual or integral form is for purposes of clarity of explanation, and not of limitation. It is also understood that the components and functionality depicted in the Figures and described herein can be implemented in hardware, software, or a combination of hardware and software.

1. A method for controlling therapy for a pulmonary disease other than a breathing rhythm disorder, comprising:
sensing one or more conditions associated with the non-rhythm pulmonary disease using sensors of a patient-external respiratory therapy device;
assessing a presence of the non-rhythm pulmonary disease based on the one or more sensed conditions; and
generating a signal for controlling delivery of a drug therapy to treat the non-rhythm pulmonary disease based on the assessment of the non-rhythm pulmonary disease.

2. The method of claim 1, wherein sensing the one or more conditions associated with the non-rhythm pulmonary disease comprises:
performing a pulmonary function test;
determining one or more pulmonary function conditions based on the pulmonary function test.

3. The method of claim 1, further comprising delivering the drug therapy using the control signal.

4. The method of claim 3, wherein delivering the drug therapy comprises delivering the drug therapy using the respiratory therapy device.

5. The method of claim 3, wherein delivering the drug therapy comprises delivering the drug therapy using a therapy device other than the respiratory therapy device.

6. The method of claim 1, wherein:
assessing the presence of the non-rhythm pulmonary disease comprises monitoring a progression of the non-rhythm pulmonary disease; and
generating the control signal for controlling the drug therapy comprises adjusting the control signal based on the progression of the non-rhythm pulmonary disease.

7. The method of claim 1, wherein assessing the presence of the non-rhythm pulmonary disease comprises:
comparing the one or more conditions to one or more sets of threshold criteria; and
assessing the presence of the pulmonary disease based on the comparison.

8. The method of claim 1, further comprising:
sensing one or more additional conditions associated with the non-rhythm pulmonary disease using an implantable device; and
assessing the presence of the non-rhythm pulmonary disease based in part on the one or more additional conditions.

9. A medical system for controlling therapy for a non-breathing rhythm related pulmonary disease, comprising:
an external respiratory therapy device, the external respiratory therapy device comprising:
a therapy unit configured to deliver respiration therapy to a patient; and
a sensor system configured to sense one or more conditions associated with a non-rhythm pulmonary disease; and
a diagnosis unit coupled to the sensor system and configured to assess a presence of the non-rhythm pulmonary disease based on the one or more sensed conditions; and
a drug therapy controller coupled to the diagnosis unit and configured to control a drug therapy delivered to the patient to treat the non-rhythm pulmonary disease.

10. The system of claim 9, wherein the sensor system comprises a ventilation airflow sensor.

11. The system of claim 9, wherein the sensor system comprises a ventilation pressure sensor.

12. The system of claim 9, wherein the sensor system comprises a ventilation gas sensor.

13. The system of claim 9, wherein the diagnosis unit is configured to perform a pulmonary function test and determine one or more pulmonary function conditions based on the pulmonary function test.

14. The system of claim 9, wherein the diagnosis unit is configured to discriminate between two or more of an obstructive pulmonary disease, a restrictive pulmonary disease, and infectious pulmonary disease, a pulmonary vasculature disease, and a pleural cavity disease.

15. The system of claim 9, further comprising an implantable device comprising an additional sensor system comprising one or more additional sensors configured to sense one or more additional conditions associated with the non-rhythm pulmonary disease, wherein the diagnosis unit is configured to assess a presence of an obstructive pulmonary disease using the one or more additional conditions.

16. The system of claim 15, wherein the implantable device comprises an implantable cardiac therapy device.

17. The system of claim 15, wherein the diagnosis unit is configured to perform a pulmonary function test using one or both of the sensor system of the respiratory therapy device and the additional sensor system of the implantable device and to determine one or more pulmonary function conditions based on the pulmonary function test.

18. The system of claim 9, further comprising a drug therapy delivery unit coupled to the drug therapy controller and configured to deliver the drug therapy to the patient.

19. The system of claim 18, wherein the drug therapy delivery unit is a component of the respiratory therapy device and the drug therapy is delivered via the respiratory therapy device.

20. A system for controlling therapy for a pulmonary disease other than a breathing rhythm disorder, comprising:
means for sensing one or more conditions associated with the non-rhythm pulmonary disease using sensors of a patient-external respiratory therapy device;
means for assessing the presence of the non-rhythm pulmonary disease based on the one or more sensed conditions; and
means for generating a control signal for controlling delivery of a drug therapy to treat the non-rhythm pulmonary disease.

21. The system of claim 20, further comprising:
means for performing a pulmonary function test; and
means for determining one or more pulmonary function conditions based on the pulmonary function test.

22. The system of claim 20, further comprising means for delivering the drug therapy using the control signal.

23. The system of claim 20, further comprising means for delivering the drug therapy using the respiratory therapy device.

24. The system of claim 20, further comprising means for delivering the drug therapy using a therapy device other than the respiratory therapy device.

25. The system of claim 20, further comprising:
means for comparing the one or more conditions to one or more sets of threshold criteria; and
means for assessing the presence of the pulmonary disease based on the comparison.

26. The system of claim 20, further comprising:
means for sensing one or more additional conditions associated with the non-rhythm pulmonary disease using an implantable device; and
means for assessing the presence of the non-rhythm pulmonary disease based in part on the one or more additional conditions.
